(19) United States
(12) Patent Application Publication
(10) Pub. No.: US 2022/0330527 A1
(43) Pub. Date: Oct. 20, 2022

(54) EQUID WEARABLE DEVICE, PERFORMANCE ANALYTICS SYSTEM AND METHODS THEREOF

(71) Applicant: HORSEPOWER TECHNOLOGIES, INC., Lowell, MA (US)

(72) Inventors: David Wayne Gilbert, Andover, MA (US); Chandra Mouli Ramani, Andover, MA (US); Victoria Brougham Thompson, South Hamilton, MA (US)

(73) Assignee: HORSEPOWER TECHNOLOGIES, INC., Lowell, MA (US)

(21) Appl. No.: 17/519,835

(22) Filed: Nov. 5, 2021

Related U.S. Application Data
(63) Continuation of application No. 16/575,146, filed on Sep. 18, 2019, now abandoned.
(60) Provisional application No. 62/732,868, filed on Sep. 18, 2018.

(51) Int. Cl. A01K 29/00; G16H 40/67; A61B 5/00; A61B 5/11

(52) U.S. Cl. CPC A01K 29/005; G16H 40/67; A61B 5/6812; A61B 5/746; A61B 5/002; A61B 5/112; A61B 5/1121; A61B 2562/0219; A61B 2503/40

(57) ABSTRACT

Systems, methods, and apparatuses for performing analytics for equine-related conditions from fetlock sensors include receiving sensor data from one or more sensors attached to one or more fetlock wearable devices. Each of the one or more fetlock wearable devices are configured to attach to a fetlock of a respective limb of a horse. The analytics system compares the sensor data to one or more baseline measurement values. The analytics system detects a condition responsive to comparing the sensor data to one or more baseline measurement values. The analytics system transmits an alert to one or more remote devices responsive to detecting the condition.

### CROSS-REFERENCE TO RELATED APPLICATIONS

This disclosure claims the benefit of and priority to U.S. Patent Application No. 62/732,868, filed Sep. 18, 2018, the contents of which are herein incorporated by reference in its entirety.

### FIELD OF THE DISCLOSURE

This disclosure relates to systems and methods for collecting and monitoring data used for evaluating an equid, such as a horse.

### BACKGROUND

Various rehabilitation, training and exercise regimens have been used to improve a horse's performance. Typically, such rehabilitation, training and exercise regimens are qualitatively analyzed and assessed to determine whether such rehabilitation, training and exercise regimens are effective. Additionally, rehabilitation is also qualitatively assessed to determine effectiveness.

### SUMMARY

The present disclosure relates to a wearable device for generating data corresponding to a horse. Such data may be used for evaluating the horse. In some instances, the data may be used for evaluating the rehabilitation, training and exercise regimens for the horse. The data may be used for early diagnosis of various conditions for the horse. The data may be used for improving the training regimen, rehabilitation strategy, etc. for the horse.

According to one aspect, a performance analytics system for monitoring a performance of a horse includes one or more servers configured to receive, via a computing device, sensor data from one or more sensors attached to one or more fetlock wearable devices. Each of the one or more fetlock wearable devices is configured to attach to a fetlock of a respective limb of a horse. The one or more servers are configured to compare the sensor data to one or more baseline measurement values. The one or more servers are configured to detect a condition responsive to comparing the sensor data to one or more baseline measurement values. The one or more servers are configured to transmit an alert to one or more remote devices responsive to detecting the condition.

In some embodiments, the one or more baseline measurement values are for a plurality of similarly situated horses. In some embodiments, the one or more baseline measurement values are for the horse at a previous point in time. In some embodiments, the fetlock wearable device is a brace including one or more motion restriction elements configured to restrict motion about the fetlock joint. In some embodiments, the fetlock wearable device is a sleeve including conductive thread. In some embodiments, the fetlock wearable device includes a sleeve with one or more sensors. In some embodiments, the condition is at least one of colic or hyper-extension of the fetlock joint.

According to another aspect, this disclosure is directed to a fetlock wearable device configured to be worn on a limb of a horse. The fetlock wearable device includes one or more sensors attached to the fetlock wearable device. The fetlock wearable device includes a communications system communicably coupled to the one or more sensors of the fetlock wearable device and an analytics system. The communications system is configured to transmit sensor data from the one or more sensors to the analytics system. The analytics system is configured to compare the sensor data to one or more baseline measurement values. The analytics system is configured to detect a condition responsive to comparing the sensor data to one or more baseline measurement values. The analytics system is configured to transmit an alert to one or more remote devices responsive to detecting the condition.

In some embodiments, the one or more baseline measurement values are for a plurality of similarly situated horses. In some embodiments, the one or more baseline measurement values are for the horse at a previous point in time. In some embodiments, the fetlock wearable device further includes one or more motion restriction elements configured to restrict motion about the fetlock joint. In some embodiments, the fetlock wearable device further includes a sleeve worn around the limb of the horse, the sleeve comprising a conductive thread. In some embodiments, the fetlock wearable device further includes a sleeve worn around the limb of the horse, the sleeve comprising the one or more sensors. In some embodiments, the condition is at least one of colic or hyper-extension of the fetlock joint.

According to another aspect, this disclosure is directed to a method for monitoring a performance of a horse. The method includes receiving, by one or more servers, via a computing device, sensor data from one or more sensors attached to one or more fetlock wearable devices. Each of the one or more fetlock wearable devices configured to attach to a fetlock of a respective limb of a horse. The method includes comparing, by the one or more servers, the sensor data to one or more baseline measurement values. The method includes detecting, by the one or more servers, a condition responsive to comparing the sensor data to one or more baseline measurement values. The method includes transmitting, by the one or more servers, an alert to one or more remote devices responsive to detecting the condition.

In some embodiments, the one or more baseline measurement values are for a plurality of similarly situated horses. In some embodiments, the one or more baseline measurement values are for the horse at a previous point in time. In some embodiments, the fetlock wearable device comprises one or more motion restriction elements configured to restrict motion about the fetlock joint. In some embodiments, the fetlock wearable device comprises a sleeve worn around the limb of the horse, the sleeve comprising at least one of a conductive thread or the one or more sensors. In some embodiments, the condition is at least one of colic or hyper-extension of the fetlock joint.

### BRIEF DESCRIPTION OF THE DRAWINGS

The disclosure will be better understood if reference is made to the accompanying drawings, in which:

FIG. 1 shows a perspective view of the range of motion limiting orthosis disclosed in Ser. No. 14/545,799, as installed over a horse's left fore fetlock;

FIG. 2 is a cross-section through the orthosis at a point where it fits around the cannon bone, illustrating the different components thereof;

FIGS. 3-8 are perspective views of a horse's right fore fetlock, illustrating the steps performed in locating the center of rotation (COR) of the fetlock;

FIGS. 9A-9E show views of the tools employed in the method of the disclosure, each being discussed separately below, these comprising a cannon tool, a pastern tool, an alignment tape, COR markers, and a measurement card, respectively;

FIG. 10 shows the use of the alignment tape;

FIG. 11 shows a perspective view of the cannon tool in use to measure the width of the left cannon bone at one of three defined distances from the COR, and includes in FIG. 11 (a) an enlarged plan view of a measurement screen;

FIG. 12A is a view comparable to FIG. 11, showing the cannon tool in use to measure the width of the left fore fetlock, and includes in FIG. 12B an enlarged plan view of the measurement screen;

FIG. 13 is a perspective view of the pastern tool in use to measure the circumference of the left fore pastern;

FIG. 14 is a perspective, partially-cutaway view of the heater to which the present application is specifically directed, the heater being used to heat the orthosis prior to final fitting to an individual, and shows the orthosis in position for being heated;

FIG. 15 is a sleeve worn on the fetlock joint;

FIG. 16 is an analytics system including a wearable device and performance analytics system for generating and analyzing measurements for a leg of a horse;

FIG. 17 is a method for analyzing sensor data corresponding to a leg of a horse; and

FIG. 18 is a communications system for providing horse-related data to interested parties.

### DETAILED DESCRIPTION

For purposes of reading the description of the various embodiments below, the following descriptions of the sections of the specification and their respective contents may be helpful:

Section A describes a device which is attachable to a fetlock of an equine.

Section B describes a computing system for generating measurements of the fetlock.

Section C describes systems and methods for generating one or more baselines for an equine.

Section D describes a performance analytics system for analytically detecting conditions for the equine.

Section E describes incorporation of 3^{rd} party data into the performance analytics system.

Section F describes a communication system.

### A. Wearable Device

In some embodiments, the present disclosure includes providing, using, or otherwise coupling an orthosis 10 to a fetlock. The method of the disclosure involves four separate steps, performed in order: location of the center of rotation (COR) of the fetlock; measurement of key dimensions of the cannon, fetlock, and pastern, at points located with respect to the COR; selection of the appropriate orthosis from a selection of models thereof; and final fitting of the selected orthosis to the individual.

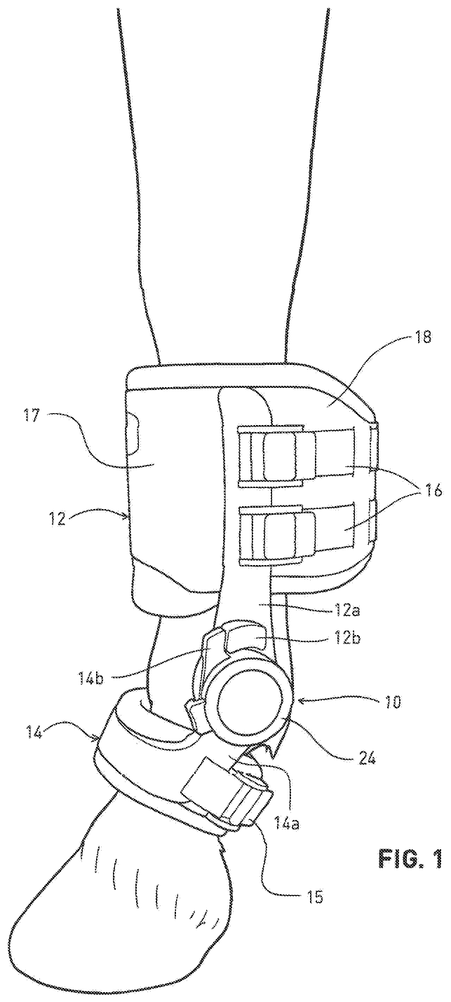

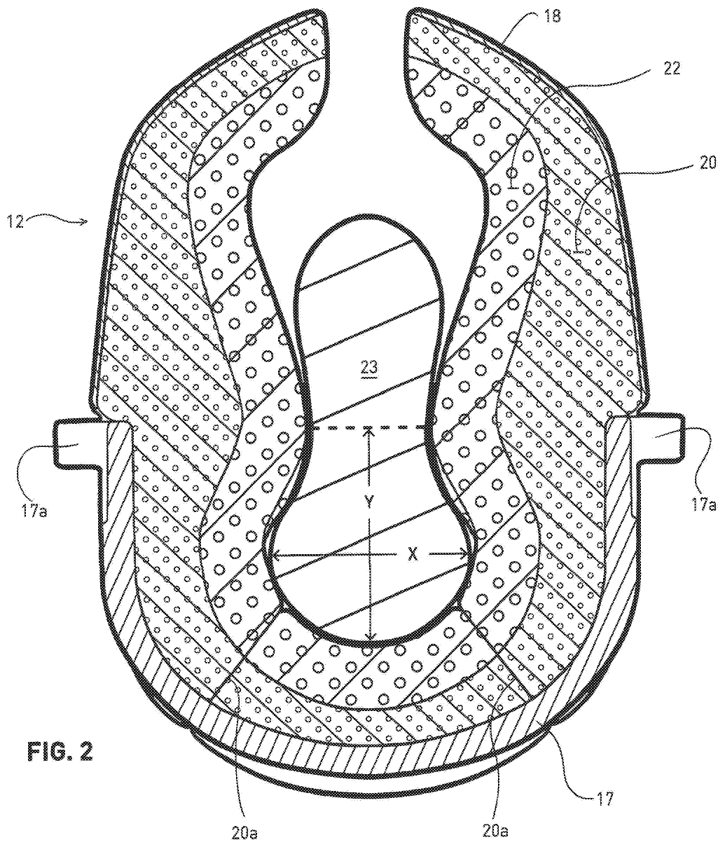

More particularly, the orthosis 10 used to limit the range of motion (ROM) of the fetlock disclosed in Ser. No. 14/545,799 (and incorporated herein by reference in its entirety) is shown in FIG. 1 affixed to the left fore fetlock region (more specifically, to the cannon and pastern) of a horse. The orthosis 10 comprises an upper or proximal cuff 12 and a lower or distal cuff 14. As currently implemented, the proximal cuff 12 comprises a hard forward shell 17 and a rear outer sheath 18 of fabric or leather. The inner padding structure comprises an outer layer 20 (see FIG. 2) of molded polyurethane (PU) foam, and an inner layer 22 of thermoformable sheet foam, such as ethylene vinyl acetate (EVA). The proximal cuff 12 is secured to the cannon bone (including in “bone” the overlying fleshy structures, skin, and coat) by straps 16. The structure of the distal cuff 14 and its affixation to the pastern bone by strap 15 are similar.

The proximal cuff 12 is pivotally secured to the distal cuff 14 by lateral members 12a and 14a fixed to the respective cuffs. The lateral members 12a and 14a meet at a pivot structure 24, which maybe as fully described in Ser. No. 14/545,799. Briefly, as the pastern rotates clockwise in FIG. 1, extending the fetlock joint, a stop 14b affixed to the distal cuff abuts a stop 12b affixed to the proximal cuff 12, limiting the ROM of the fetlock. The relative position of one or the other of the stops can be varied to limit the ROM to a desired degree. Again, see Ser. No. 14/545,799 for a preferred structure permitting this adjustment to be readily accomplished. Not seen in FIG. 1 are medial members corresponding to lateral members 12a and 14a which meet at a similar pivot structure, but lack the ROM stop mechanism, which is provided only on the lateral side of the orthosis 10.

The right-side orthosis is a mirror-image of that shown in FIG. 1. As noted, the pivot structures 24 allowing adjustment of the ROM of the fetlock are placed on the laterally-outer sides of the fetlocks, to avoid interference that would likely occur if this protruding structure were disposed on the medial inner side of the fetlock, especially noting that the orthoses are typically employed in pairs.

It will be apparent that in order to provide the maximal therapeutic function the cuffs must fit their respective bones closely and securely, so as to avoid slippage, and that the COR of the pivot structure of the orthosis must be substantially aligned with the COR of the fetlock, so as to achieve friction-free rotation and avoidance of unnatural pivoting of the fetlock.

The present disclosure is directed to achieving the good fit and accurate alignment mentioned above while providing the orthosis in a readily manufacturable form at reasonable cost. That is, although it would theoretically be possible to custom-fit a unique orthosis to each horse to be treated, this would be very time-consuming and inefficient. Moreover, the time taken to manufacture such a custom orthosis for a given horse might interfere with healing; that is, it would be preferred to have a number of premanufactured orthoses on hand for custom-fitting in a rapid fashion, so as to obtain the therapeutic effects thereof as rapidly as possible. An important aspect of the disclosure is therefore to provide a method for expeditiously determining which of a plurality of premanufactured orthoses is the best fit for a particular horse, and then to provide a method for rapidly custom-fitting the orthosis to the horse. However, as indicated above, the tools disclosed herein and employed for selecting the correct orthosis from a selection thereof could also be employed for making measurements useful in making custom-made orthoses.

As noted above, referring to FIG. 2, the proximal cuff 12 fitting over the cannon bone, shown approximately as a hatched section 23, comprises a forward shell 17 formed of plastic or metal, to which the straps 16 are attached, and to which the medial and lateral members 12a and 14a are riveted, and comprising bump-outs 17a on either side for alignment of the medial and lateral members, a thinner rear sheath 18 of fabric or leather, a first layer 20 of foam, e.g., polyurethane (PU) that is molded to define the basic inner contour of the cuff in contact with the cannon bone, and a second layer 22 of thermoformable sheet foam, of uniform thickness, and made of ethylene vinyl acetate (EVA) or the like. The foam layers may be made in several portions, as illustrated, and assembled with adhesive. The combination of the forward shell 17, rear sheath 18, and the molded PU layer 20 together define the “model” of the cuff, which is selected in response to the detailed measurement techniques described below. The cuff 12 is then custom fit to the horse by heating it, preferably in the specialized heater claimed in this application, until the EVA layer 22 is warmed sufficiently to be formable. The cannon cuff 12 is then placed quickly over the cannon bone and the straps 16 tightened. The pastern cuff 14 is fit similarly and simultaneously. As the EVA cools it hardens, so that its surface conforms to the outer surface of the respective bones. The heat content of the EVA is low, so that the horse is not burned painfully in the process. It should also be understood that a generally comparable technique employing a thermoformable foam is used for fitting ski boots to skiers' feet.

More specifically, the padding consists of two layers, an outer polyurethane (PU) foam layer 20 and an inner thermoformable foam layer 22. The PU foam layer 20 is injection-molded to define the shape of the inner contour of the cuff in a flat configuration with webs between the three sections in which it is molded, as indicated at 20a. The webs are either made sufficiently flexible that the PU layer 20 can be folded into its final shape, or the webs are removed and the parts are separated for later reassembly. The thermoformable foam layer 22 is cut to shape and then heated and compression molded so as to follow the contours of the PU foam layer 20. The PU foam layer 20 and the thermoformable foam layer 22 are then laminated together using adhesive.

In order to prevent the top and bottom edges of the thermoformable foam layer 22 from flattening out during the heating and fitting process for the horse, its edges are stitched to small injection-molded pieces of elastomeric thermoplastic polyurethane (TPU) termed welts (not shown). Therefore, the complete process of assembling the thermoformable foam layer 22 is to (a) cut out the thermoformable parts, (b) stitch them to the welts and (c) laminate the welts and the thermoformable foam to the PU foam using adhesive. When the orthosis is fitted to the horse, the thermoformable foam maintains its outer contour due to the lamination but the inner contour changes to replicate the anatomy of the horse.

The provision of tooling to form the forward shell 17 is the most costly part of arranging for manufacture of the orthosis. Research has shown that the vast majority of horses can be accommodated with left and right shells 17 in a single size. The molded PU foam then defines the basic fit of the cuff over the cannon bone. Again, research has shown that the vast majority of horses can be accommodated if the molded PU is provided in four widths, dimension X in FIG. 2, where X is the maximum interior transverse dimension of an approximately oval forward section of the cuff, and two lengths, dimension Y in FIG. 2, the fore and aft dimension between the forwardmost surface of the oval forward section of the cuff and its narrowest point. Accordingly, 16 possible proximal cuffs are provided: 4 widths×2 lengths×2 (for left and right).

It has further been determined that there is some variation from horse to horse in the way in which the width of the cannon bone varies along its axial length. Therefore, as will be explained further below, its width is measured at three locations spaced from the COR, and the widest selected for the width X.

The distal pastern cuff 14 is structured and fit similarly, and is provided in 4 sizes, selected responsive to measurement of the circumference of the pastern at a given distance from the COR.

The medial and lateral members 12a and 14a are also provided in differing widths, corresponding to the width of the distal pastern cuff 14.

Thus a total of 128 models of the orthosis (8 proximal cuffs×4 distal cuffs×2 for left and right and a wide and narrow size) is sufficient to fit the vast majority of horses.

Turning now to the method of fitting the orthosis to the horse, the first step is to locate the center of rotation (COR) of the fetlock, so as to ensure that the COR of the orthosis is correctly aligned with that of the fetlock. The COR is also used as the reference point from which the locations for most of the measurements needed are taken. The steps described in the following are but one way to locate the COR, and other methods of doing so are within the scope of the disclosure.

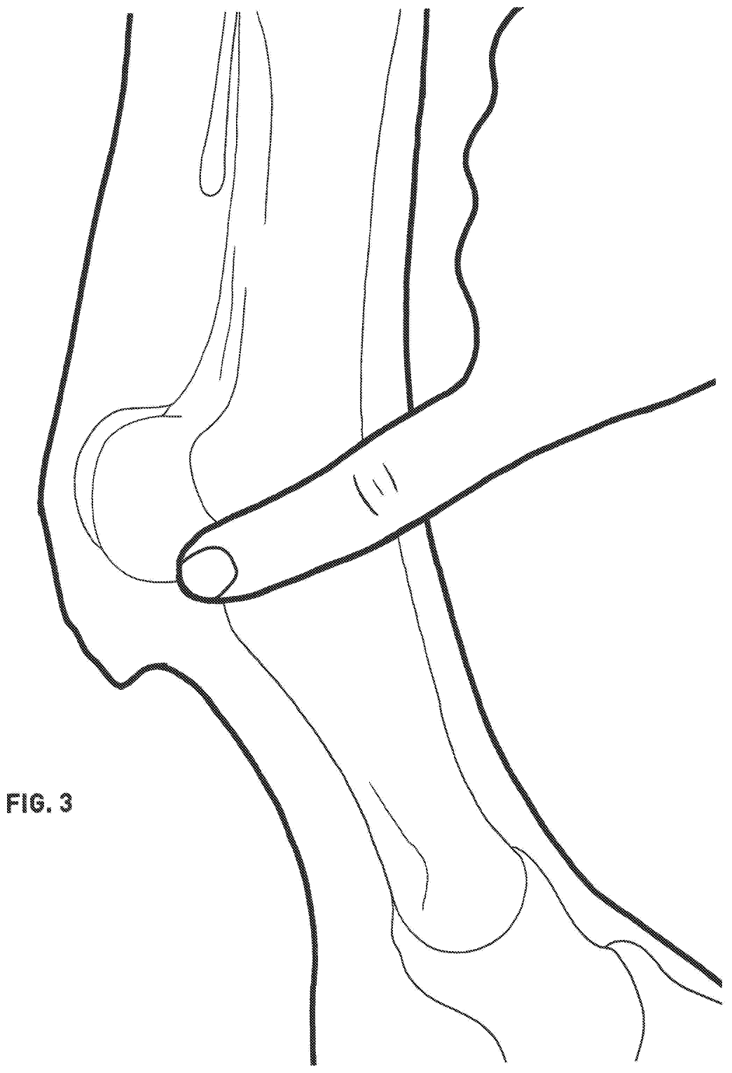

The first step is shown in FIG. 3, which illustrates the horse's right foreleg, with the bone contours shown by lighter weight lines. With the horse standing still on a flat firm surface, the user palpates the fetlock with the index finger and locates the depression between the palmar process of the first phalanx and the base of the ipsilateral (same side) proximal sesamoid bone. This can be identified as feeling like a “divot” on the surface of the fetlock.

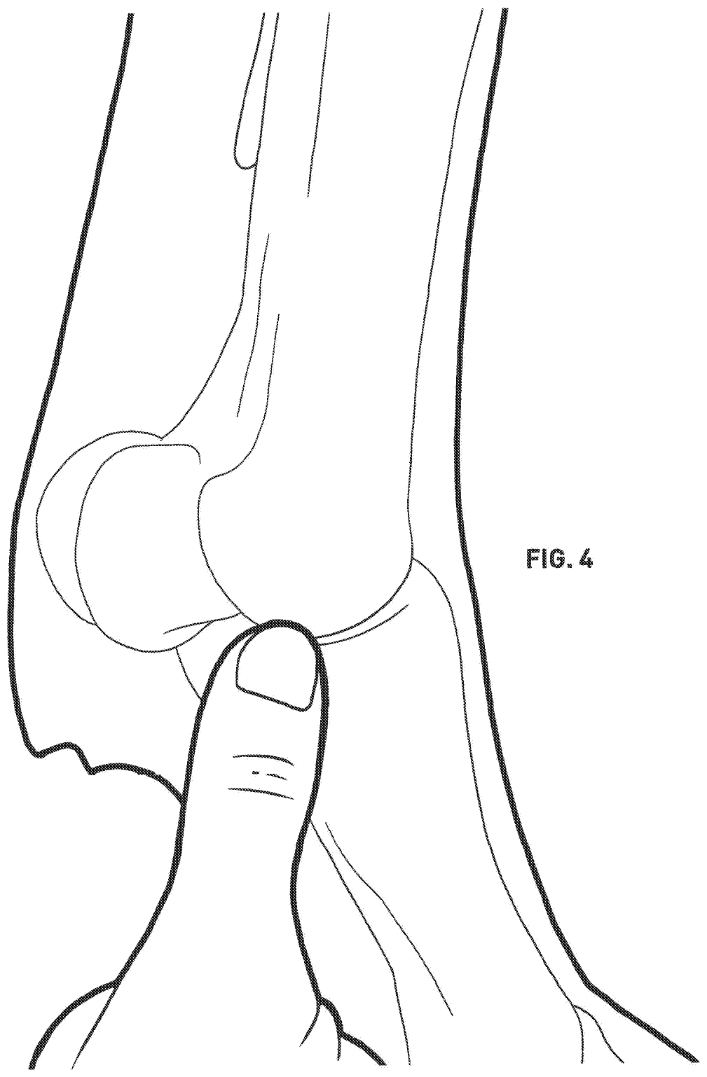

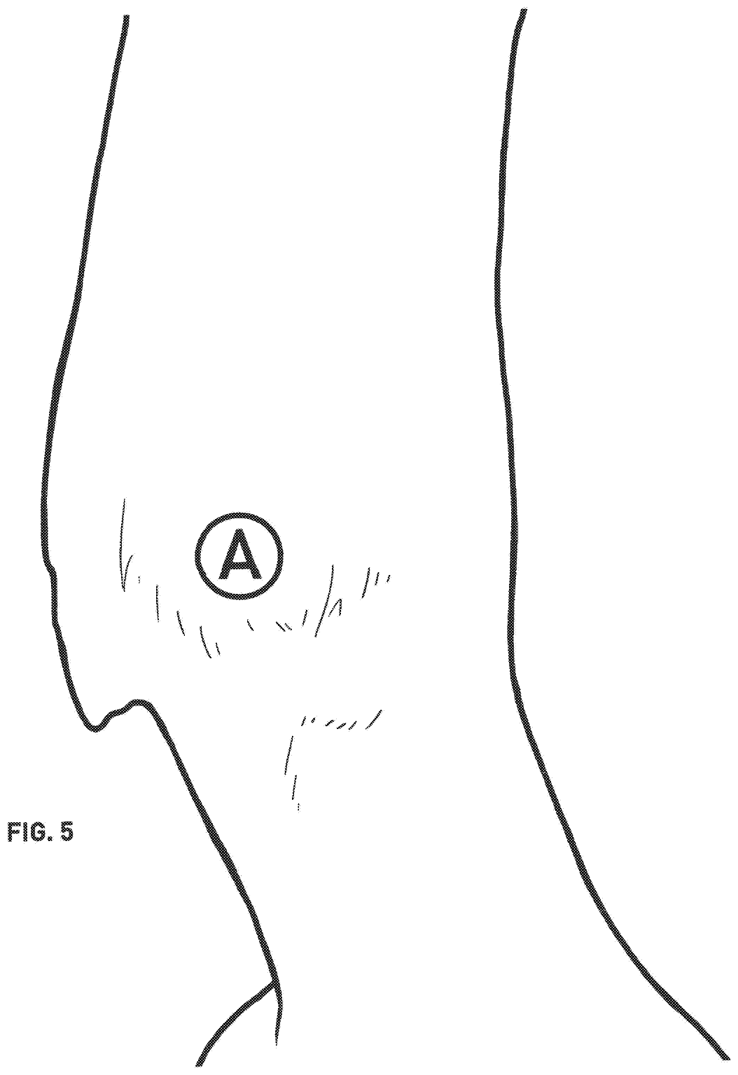

Next, as shown in FIG. 4, the user employs a thumbnail to identify the palmar-most (toward the rear of the horse) joint margin. As shown in FIG. 5, an adhesive marker, identified as marker A, is then applied to the joint at this point.

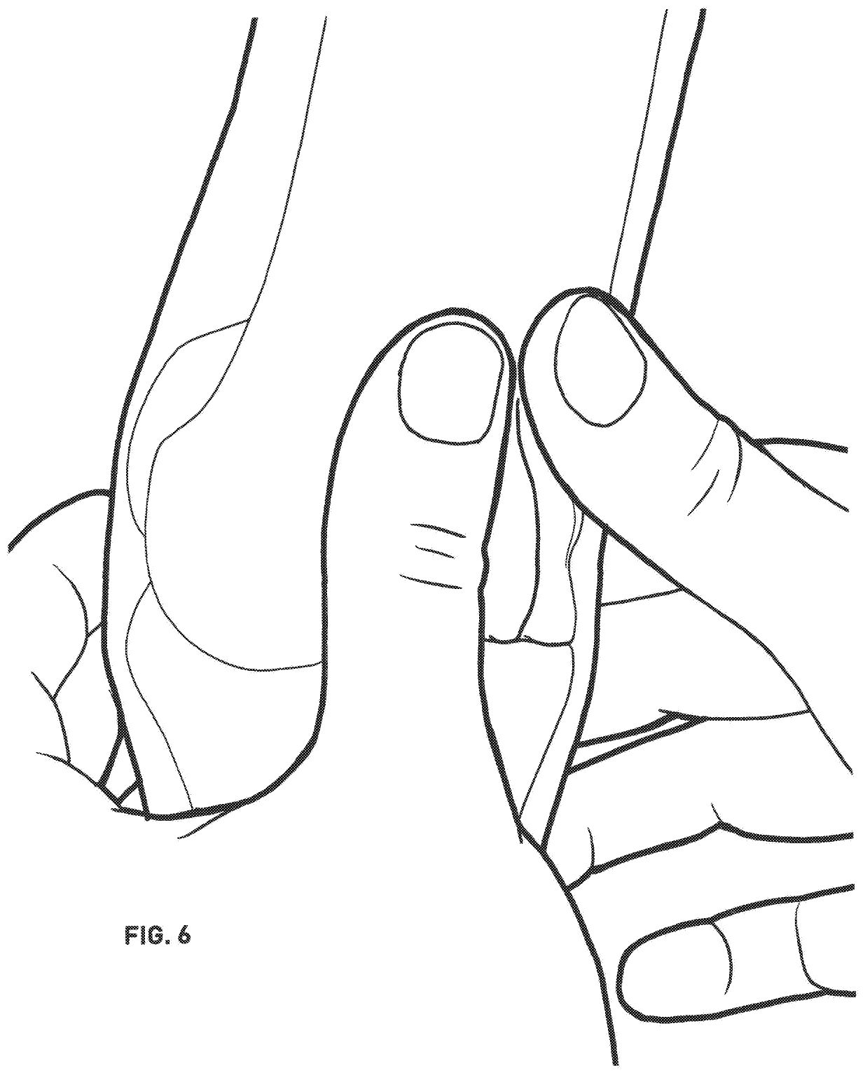

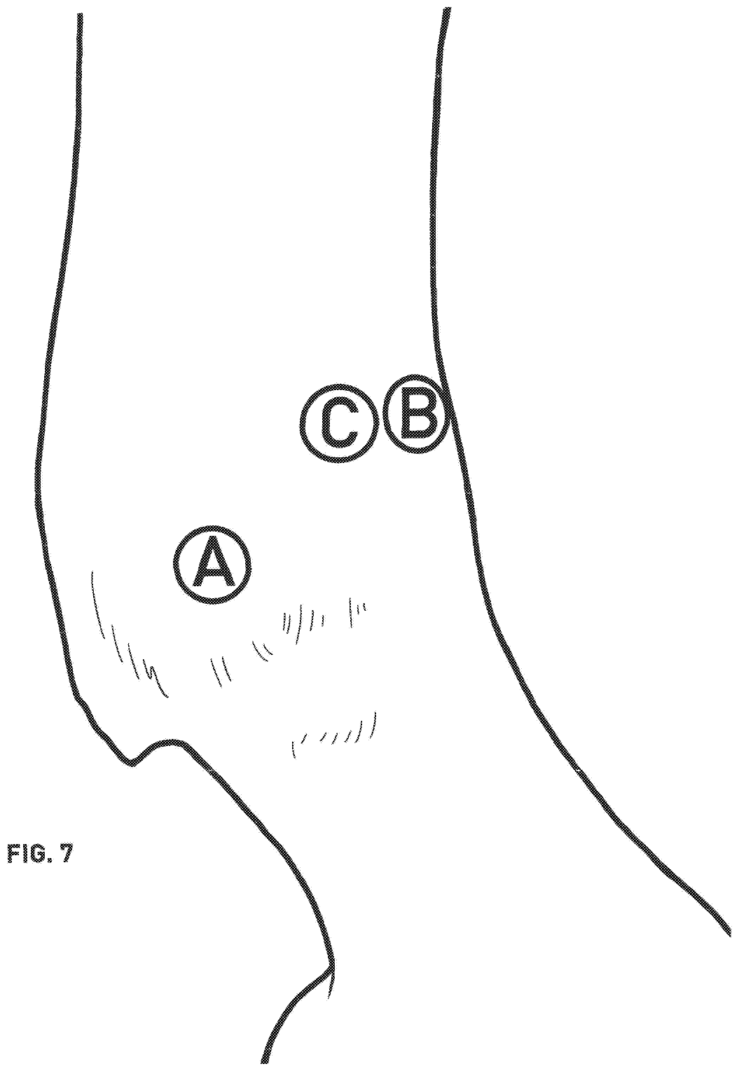

Next, as illustrated by FIG. 6, the user identifies the proximal-most prominence of the intercondylar ridge on the cranial aspect of the cannon near the fetlock. A marker B is placed where the intercondylar ridge merges with the flat cranial surface of the distal cannon bone. This point is identified by deeply palpating the front of the lower cannon bone with both thumbs, as illustrated. After marker B is placed at this point (see FIG. 7), a second marker C is placed at the same level with respect to the horizontal, but on the forward-most part of the lateral surface of the cannon bone. Again, see FIG. 7. Marker B can then be removed.

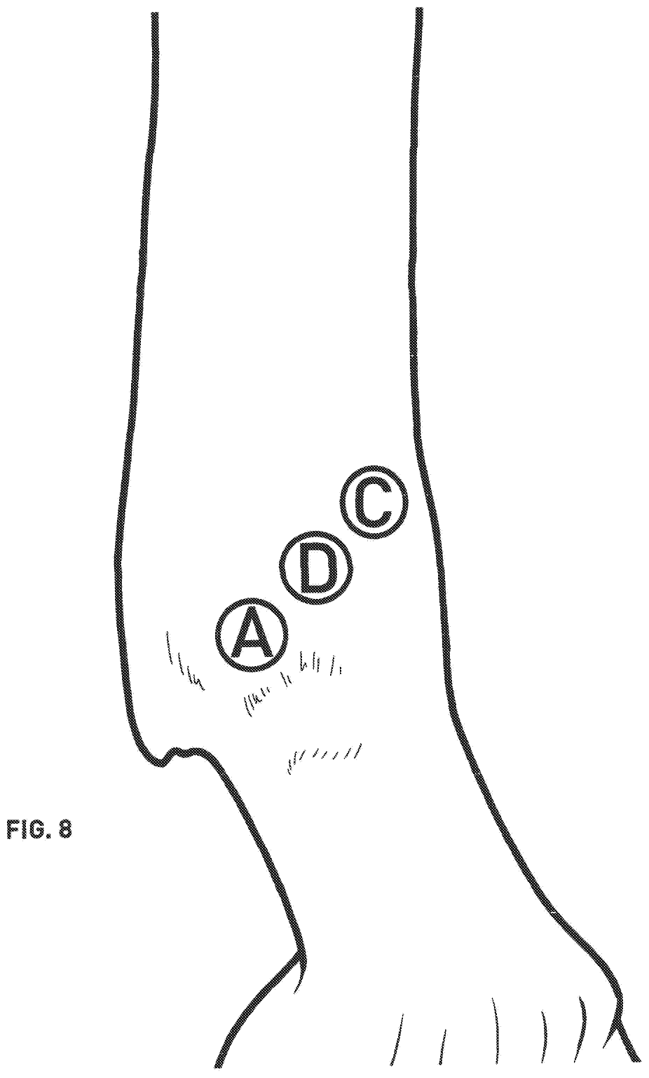

Finally, a fourth marker D is placed is placed midway between markers A and C, as illustrated by FIG. 8. This is the center of rotation (COR) of the fetlock. Markers A and C can then be removed.

The COR of the fetlock having thus been located, measurements can be taken using the COR as a “base point” from which the other measurement are located, ensuring that the orthosis thus fitted will have its COR substantially aligned with the COR of the fetlock.

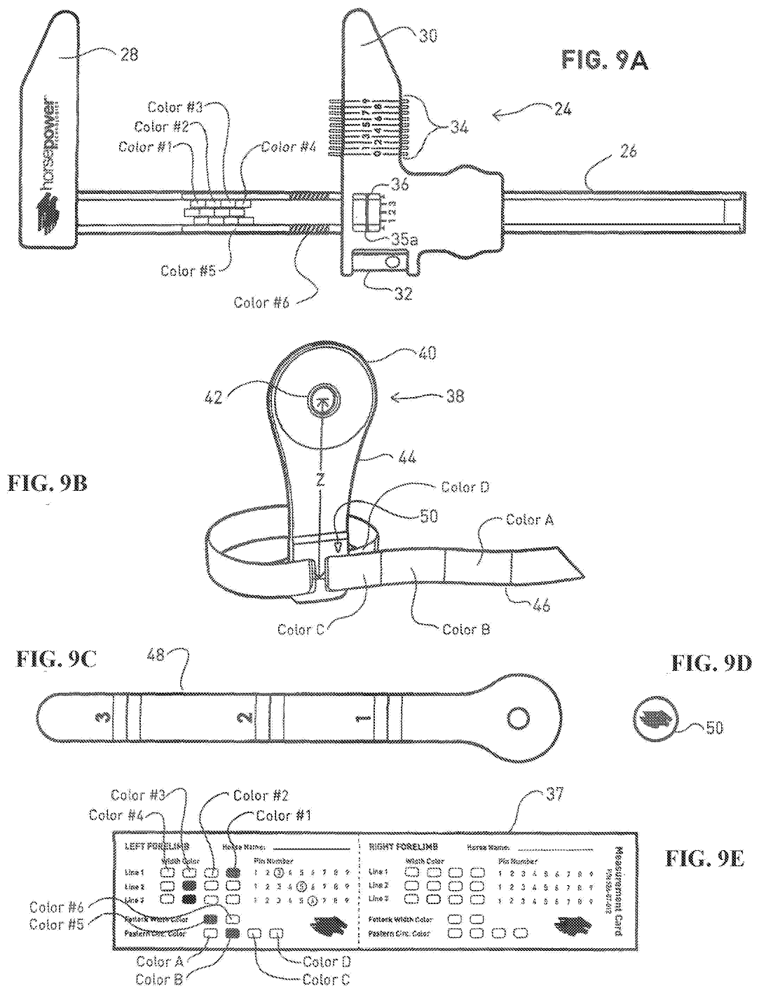

FIGS. 9A-9E show a kit of tools provided by the proprietor of the orthosis to ensure proper fitting of the orthosis to the fetlock. It will be appreciated by those of skill in the art that comparable measurements could be made using different tools; those shown are but one convenient possibility. Further, several different embodiments of the tools shown could be employed; these will be discussed as appropriate.

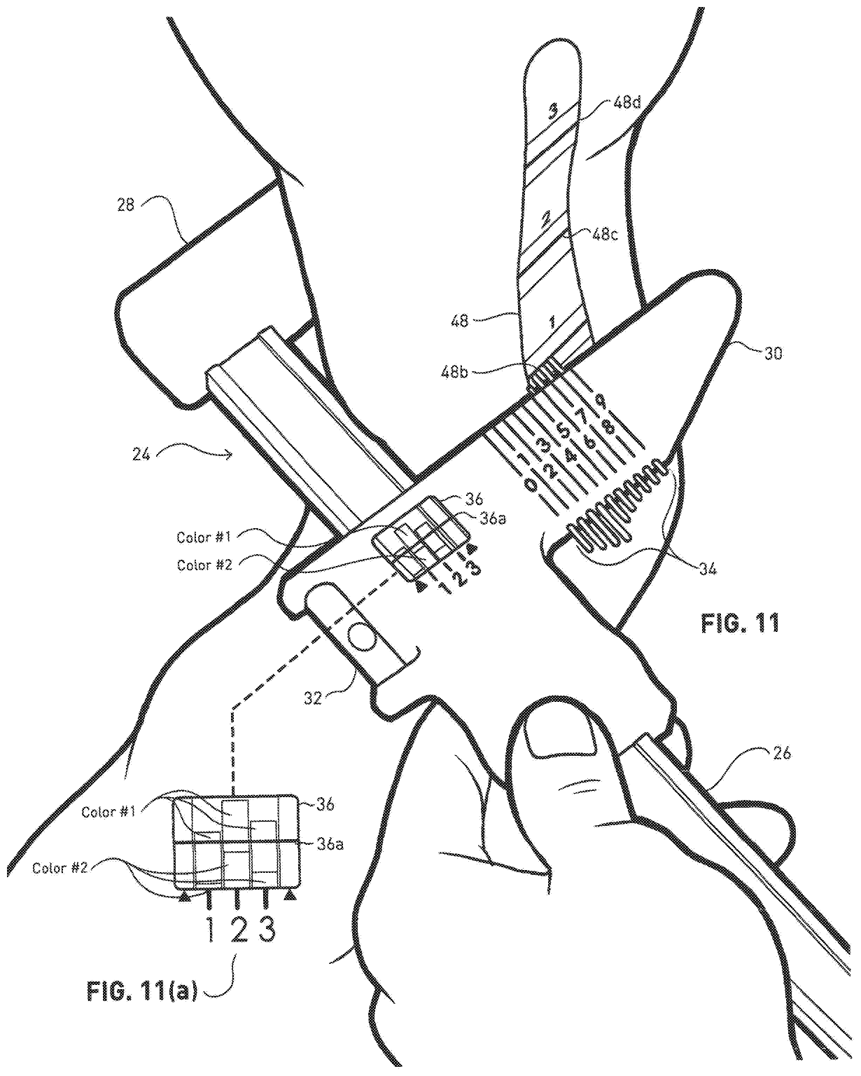

The cannon tool 24 shown in FIG. 9A is used to measure the width X of the cannon bone and to locate the distance Y between the front of the cannon bone and its point of maximal width, which are important in selecting the proper model of the proximal cuff, as described above with reference to FIG. 2. The cannon tool 24 resembles a caliper, comprising a beam 26, a first anvil 28 fixed to one end of the beam 26, and a second anvil 30 sliding along beam 26. As illustrated by FIG. 11, and more fully discussed below, in order to measure the width of the cannon bone, the fixed anvil 28 is juxtaposed to one side of the cannon bone, with the beam held horizontal (as may be confirmed using a bubble level 32 mounted to the sliding anvil 30), in contact with the cannon bone, and square to the horse's centerline. The sliding anvil 30 is then brought into contact with the opposite side of the cannon bone. The distance between anvils 28 and 30 is then equal to the width X of the cannon bone. At the same time, a plurality of numbered pins 34 sliding in bores in sliding anvil 30, and spring-biased toward the inner surface of sliding anvil 30, that is, in the leftward direction in FIG. 9A, are brought into contact with the outer surface of the cannon bone. These pins are numbered, as indicated. One of the pins, located over the widest portion of the cannon bone, will protrude more than the others; its number is noted and used to specify the depth Y of the widest point of the cannon bone from its forward surface.

The distance X between the anvils during the measurement process may be determined in a variety of ways; for example, the beam 26 could be inscribed with inch or metric indicia, as in a conventional caliper. However, for reasons of convenience to the user, color-coded marks indicated by “colors 1— 6” are printed on beam 26 of the cannon tool 24. A window 36 is formed in the sliding anvil 30, with a reference line 36a provided thereon. When a measurement is made, the color of the mark under the reference line 36a is noted, and a measurement card 37 shown in FIG. 9E marked accordingly. The number of the pin that protrudes outwardly more than the others is also noted. The color-coding scheme employed in the preferred embodiment is described in connection with FIG. 11, below, as are details of the measurement process.

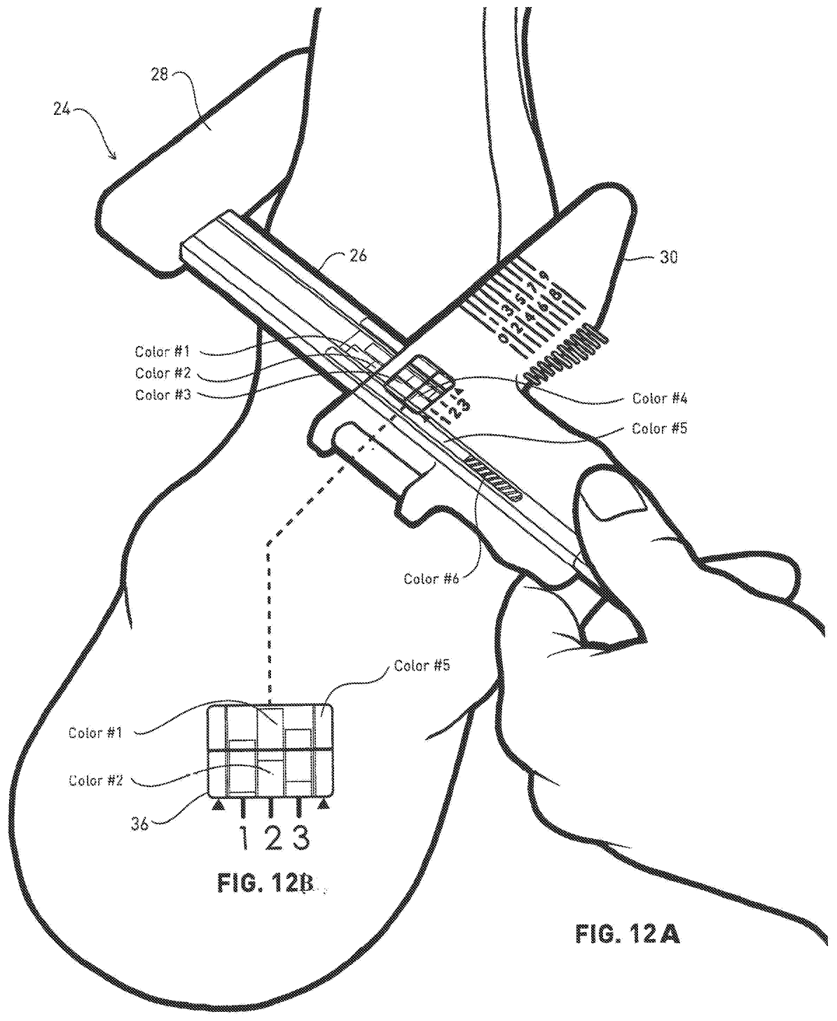

The cannon tool 24 is also used to measure the overall width of the fetlock, as described in connection with FIG. 12A below; this measurement is used to determine whether the orthosis is wide or narrow, that is, whether wide or narrow medial and lateral members 12a and 14a are needed.

The cannon tool 24 is provided with a second window on its opposite side, and the beam provided with a second set of colored marks, so that the tool 24 can be flipped over and used to make similar measurements of the opposite leg.

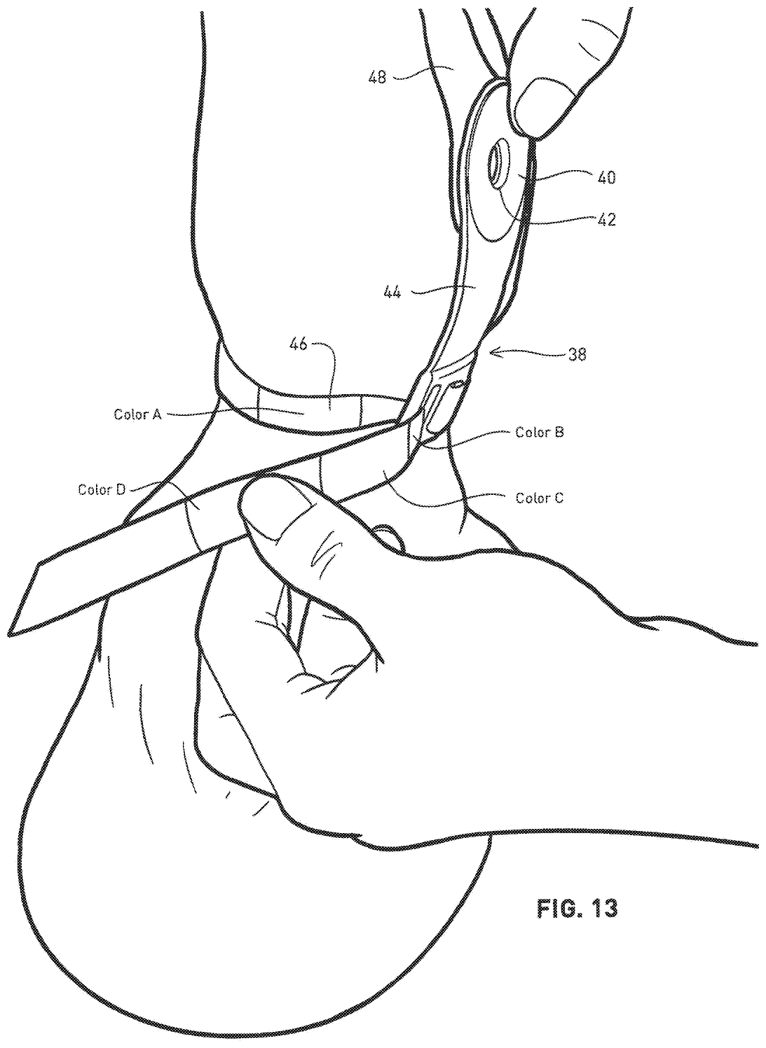

As discussed briefly above, the circumference of the pastern is measured in order to determine the proper combination of molded PU and thermoformable sheet foam to be provided in the distal cuff. A pastern tool 38, shown in FIG. 9B, is provided for the purpose. This comprises a circular head portion 40 having an aperture 42 at its center. The pastern tool 38 is disposed on the pastern so that aperture 42 is located directly over the COR of the fetlock, that is, tool 38 is located so that marker D (FIG. 8) is disposed within aperture 42. A tongue 44 depends from head member 40, and a measuring ribbon 46 is secured thereto at a distance Z from the center of aperture 42. In use the ribbon 46 is passed around the pastern and the length of the ribbon 46 needed to circumscribe the pastern is noted. Again, this measurement could be made using conventional inch or metric indicia, but is preferably implemented using a color-coded system, as further detailed in FIG. 13 below.

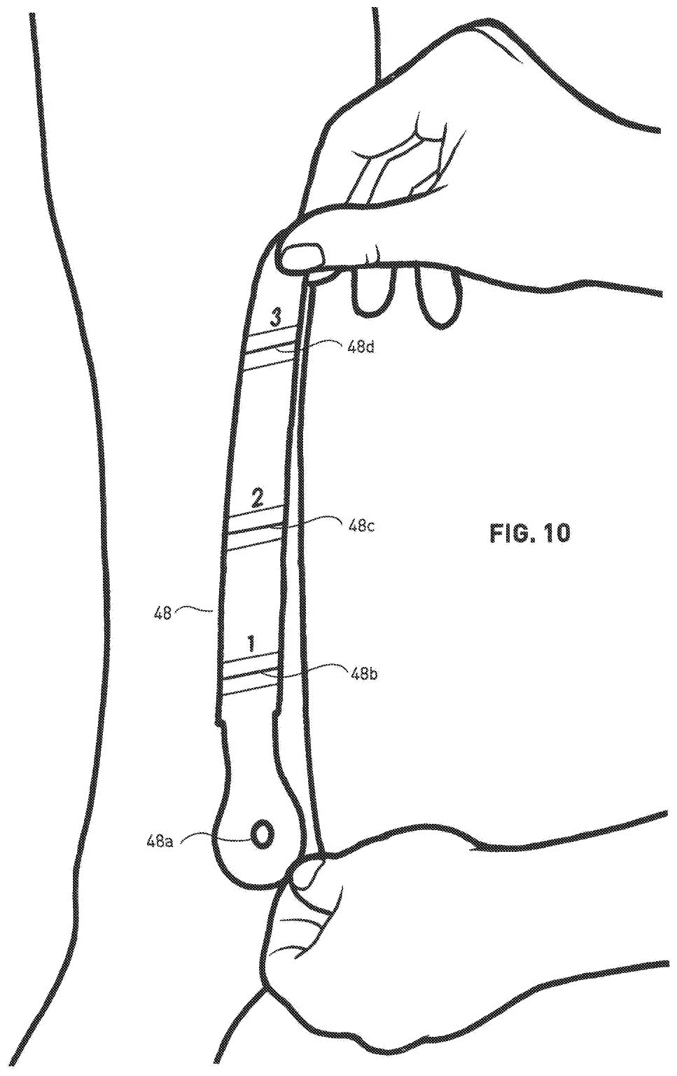

FIG. 9C shows an alignment tape 48 that is employed to locate three distances from the COR along the axial extent of the cannon bone at which measurements of the width and length of the cannon bone are made, as detailed below in connection with FIGS. 10 and 11. Tape 48 has an aperture 48a that in use is located over the COR of the fetlock. Tape 48 has an adhesive backing for allowing it to be conveniently secured to the cannon bone. A ring of hook and loop fastening material, nonwoven fabric or the like is preferably provided around the aperture 48a for attachment of the pastern tool 38, which is provided with a mating ring of mating material.

FIG. 9D shows one of the adhesive markers 50 that are used in determination of the COR, as described above.

Finally, FIG. 9E shows a measurement card 37 which provides printed spots which can be darkened with a pen or marker to record the width measurements in a convenient, easy-to-use manner, numbers that may be circled to identify the pin noted in the depth measurement, a space for provision of horse identification data, and the like. After the measurements are recorded, card 37 may be sent to the proprietor of the orthosis for selection of the correct model, or may be used as part of a paper-based, online or electronic selection method.

The measurement process begins as illustrated by FIG. 10, showing that the alignment tape 48 is secured to the cannon bone such that marker D, locating the COR as discussed above, appears within an aperture 48a in the alignment tape 48. The alignment tape 48 is also preprinted with markings 48b-d indicating predetermined distances from the COR at which the measurements of the cannon bone's width and depth are made; these are referred to as positions 1-3.

FIG. 11, including an enlarged version of the window 36 as FIG. 11(a), illustrates the process of simultaneously measuring the width and depth of the cannon bone. As discussed above, the cannon tool 24 is brought into contact with the cannon bone such that beam 26 contacts the forward surface of the cannon bone at a predetermined distance above the COR, as indicated by the alignment tape 48; in the drawing, the cannon tool 24 is being used to take measurements at position 1 on the alignment tape 48, as indicated by marking 48b. The cannon tool 24 is held level, employing level 32 to confirm this, and square to the central axis of the horse. The anvils 28 and 30 are brought into contact with medial and lateral surfaces of the cannon bone, such that the distance between the anvils is equal to the width X of the cannon bone at position 1. As noted above, this distance could be measured directly using inch or metric markings, but is preferably simply recorded as a color value.

More particularly, as illustrated in FIG. 9A, the beam is provided with three sets each of four colored areas, corresponding to positions 1-3 on the alignment tape. These are indicated as “colors 1-4”, as colors cannot be used in patent drawings; in the preferred embodiment, these are four different colors. When a measurement is made, the color under the line 36a in window 36 corresponding to the position at which the measurement is made is noted, and the corresponding spot on the measurement card 37 darkened. In the example shown in FIG. 11(a), color #1 is under the line 36a opposite the marking corresponding to position 1, and the corresponding spot on the measurement card 37 in FIG. 9E has been darkened.

At the same time, the spring-biased pins 34 are in contact with the lateral outer surface of the cannon bone, and one of these will protrude more than the others, corresponding to the depth of the cannon, that is, its widest point. In FIG. 11, this is pin 3. The corresponding pin number has been circled on the measurement card 37. It will be appreciated that the pins 34 could be omitted, and the sliding anvil 30 be provided with numbered markings corresponding to the numbers of the pins shown, so that the depth of the maximum width of the cannon bone could be identified by noting the marking corresponding thereto, e.g., by eye or touch. However, the pins 34 make this identification more positive.

It will be appreciated that the cannon tool 24 is thus capable of making measurements in two dimensions simultaneously, that is, the width X of the cannon bone and the depth Y at which its maximum width is located.

The same procedure is then repeated at positions 2 and 3 as defined by markings 48c and 48d on the alignment tape 48, and the results recorded similarly on the measurement card 37.

As illustrated, the positions of the colors on the beam are offset with respect to one another at positions 1, 2 and 3. This is done corresponding to the variation in width of the cannon bone with distance from the COR; the cannon bone narrows near its midpoint as compared to its ends.

The cannon tool 24 is then used to measure the width of the fetlock by placing the opposed anvils against the fetlock at the height of the COR, as illustrated in FIG. 12A, including an enlarged view of the window 36 in FIG. 12B. In this case, the width is measured by noting the position of line 36a to one of two colors, #5 and #6, provided along the edges of the beam 26, as shown in FIG. 9A. In the example of FIG. 12A, the line 36a is disposed over color #5, and the corresponding spot on the measurement card of FIG. 9E has been darkened. This measurement is used to determine whether the orthosis is to be wide or narrow.

The final step in taking the measurements is measurement of the pastern circumference. This is done as illustrated in FIG. 13. The pastern tool 38 described above is affixed to the alignment strip 48 so that the aperture 42 in the pastern tool is disposed over the COR; mating hook and loop fasteners or the like may be provided thereon for convenience. The tongue 44 extends downwardly, over the fetlock, defining the distance Z between the COR and the point on the pastern at which the circumference is measured. The ribbon 46 is pulled around the pastern snugly. Ribbon 46 is provided with four colored sections, A-D, as indicated. That which is located opposite a marker 50 (FIG. 9B) is taken as the measurement, and is recorded on the measurement card 37. In the example of FIG. 13, color B is thus chosen, and the corresponding spot on measurement card 37 has been darkened.

The same process is then performed on the other leg, as the orthoses are generally used in pairs. As noted, the cannon tool is provided with measurement windows and colored patches on both sides, so that the tool can simply be flipped over and used on the opposite leg. As shown by FIG. 9E, the measurement card 37 is provided with duplicate spots for entry of the same measurements for both legs.

The measurement card 37 is then, for example, forwarded to the provider of the orthoses, who chooses the appropriate orthoses from the stock of models and provides these to the user, typically a veterinarian. Other options include ordering the orthoses employing a manual look up table, a phone app, or an online selection webpage. As discussed above, where the width of the cannon bone varies along its length, the maximal width is used to select the correct orthosis.

The final step is fitting the orthosis to the individual. As noted above, the measurement steps above are used to select the closest-fitting orthoses from a considerable number of models. The final fitting is performed by heating an inner layer 22 (FIG. 2) of a thermoformable foam material, for example ethylene vinyl acetate (EVA), of the proximal and distal cuffs, to the point that it can be compressed around the cannon and pastern bones, and clamping the orthosis on the fetlock in place using the straps 15 and 16. As the EVA cools it takes the shape of the cannon and pastern bones, ensuring a very good fit of the orthosis to the fetlock.

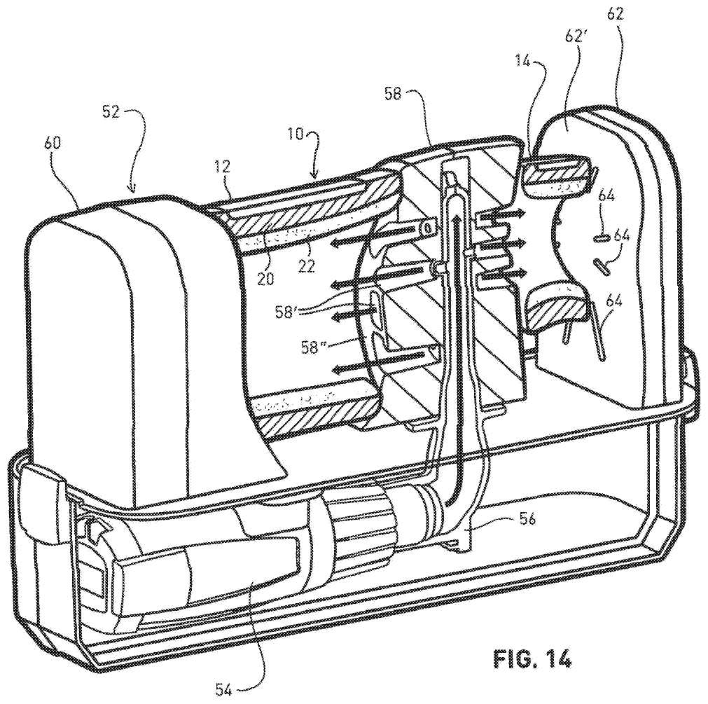

FIG. 14 shows a heating device 52 according to the present disclosure that is particularly adapted for heating the orthosis as described above. Heating device 52 comprises a heating assembly 54 of a heating element and a fan, providing a stream of hot air via ducting 56 to a perforated plenum 58 defining a number of outlet ducts 58′, which provide a number of air streams indicated by arrows in FIG. 14. In use, the orthosis 10 is placed over the plenum 58, so that the cannon cuff 12 is confined between plenum 58 and a first platen 60, and the pastern cuff 14 between plenum 58 and a second platen 62, defining substantially closed cavities. The width of the plenum is selected in correspondence with the space between the cannon and pastern cuffs defined by the pivot structure. All of the various sizes of the orthosis have the same longitudinal dimensions, so that the same heater can be used to fit any size of orthosis. However, it would be within the skill of the art to make the platens relatively movable with respect to the plenum if it were desired to accommodate orthoses of differing dimension or to change the degree of sealing between the corresponding surfaces. Plenum 58, platens 60 and 62, and ducting 56 may all be molded of glass-fiber reinforced nylon, of the grade known in the art as nylon 6, 6.

The hot air heats the EVA foam 22 to a desired temperature, typically 250° F., at which point the orthosis 10 can be removed from the heating device 52 and promptly clamped around the fetlock, as described above, so that the EVA layers 22 in the proximal and distal cuffs conform to the shapes of the cannon and pastern, respectively. The temperature of the surface of the EVA layers 22, and/or the air temperature within the inner cavities may be measured and used to control the operation of the heating assembly, or a timer may be employed to ensure adequate heating.

Geometric features, such as ribs 64, are shown on the inner surface 62′ of platen 62, juxtaposed to the pastern cuff 14. These features, which if implemented as ribs 64, may be on the order of ⅛-¼″ in height, space the end of the pastern cuff 14 from the platen 62, providing a controlled exit for air flowing from plenum 58, that is, between the end of the generally cylindrical pastern cuff 14 and platen 62. Similar geometric features (not shown) may be provided for the same purpose on the surface (not shown) of platen 60 juxtaposed to the cannon cuff 10, and on the surface (not shown) of plenum 58 juxtaposed to the pastern cuff of the orthosis 10. However, in a preferred embodiment, no such features are provided on the surface 58″ of the plenum 58 juxtaposed to the cannon cuff 12. Thus, in this embodiment the surface 58″ of the plenum 58 is relatively sealed to the cannon cuff 12, while the surface of the cannon cuff juxtaposed to the platen 60 is spaced therefrom by ribs 64, and the surfaces of plenum 58 and platen 62 are both spaced from the pastern cuff 14, providing controlled leakage of hot air flowing from plenum 58. In general, all of the surfaces that are juxtaposed to the orthosis during the heating step may or may not have geometric features as needed to govern the flow of air in order to produce relatively uniform heating. The contoured shapes of the plenum and platen surfaces relative to the mating contours at the ends of the padding also control the amount of air leakage. In order to limit the escape of hot air from the openings at the rear of the cuffs that are necessary to allow the orthosis to slip over the fetlock, these openings may be closed during heating using the straps and overwrapped with Velcro closures. However, the hot air flows at sufficiently high velocity from ducts 58′ that most of the flow is in the vicinity of the inner surface of the cuffs, providing efficient heating.

Noting that the interior volume of the cannon cuff 12 is substantially greater than that of the pastern cuff 14, due to their differing axial lengths, the differing degrees of sealing thus provided, together with the detailed design of ducts 58′ in plenum 58, are cooperatively selected so as to control the flow of air from plenum 58 via ducts 58′ so that the flow of air from heating assembly 54 substantially uniformly heats the interior surfaces of thermoformable foam layers 22 of the cannon and pastern cuffs, so that when the orthosis is subsequently clamped over the fetlock the thermoformable members 22 thereof are substantially uniformly formable over the respective leg geometry.

It will be appreciated that by fitting closely over the heating device 52, with the cannon and pastern cuffs in substantially sealed relation with plenum 58 and platens 60 and 62, the orthosis 10 essentially provides two substantially closed volumes over the plenum 58, one each within the volume defined by the cannon and pastern cuffs. In this way, the hot air heats only the interior EVA surface of the cannon and pastern cuffs. By comparison, if the orthosis were to be heated, for example, in an oven, it would be heated throughout, including its exterior surface, which would be inconvenient for handling, and would require a great deal of additional energy. Similarly, heating the orthosis by supplying hot air to one end would not promote uniform heating of the inner surface.

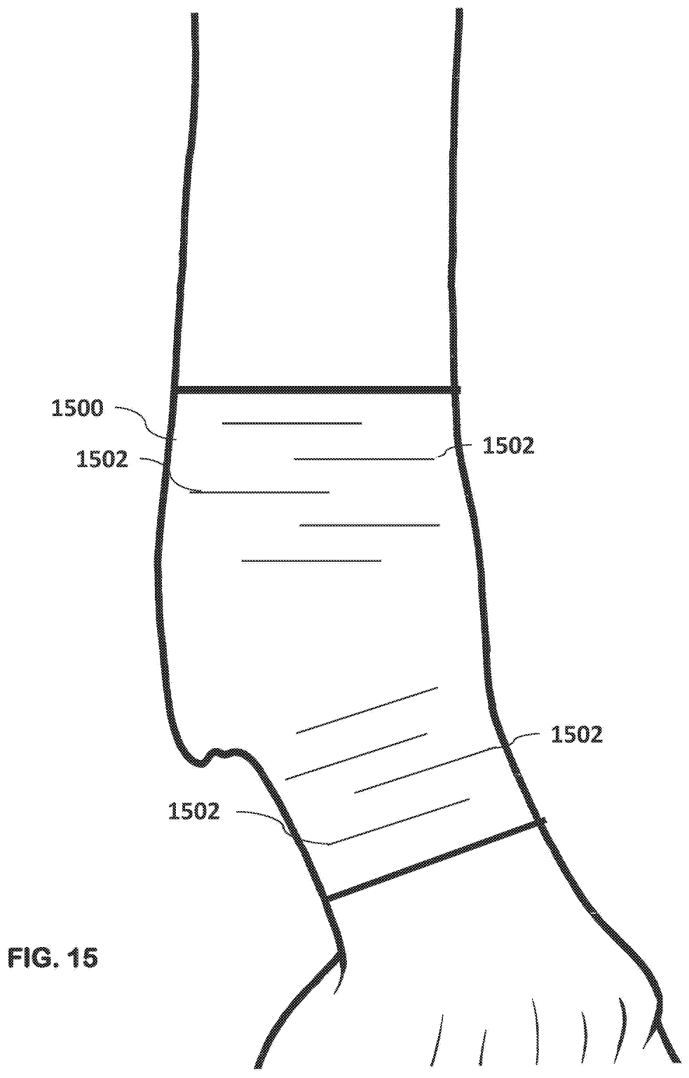

Referring now to FIG. 15, a sleeve 1500 (or wrap) worn on the fetlock, according to an exemplary embodiment. In some implementations, the sleeve 1500 may surround the perimeter of the fetlock joint, and may extend upwardly towards the cannon, and downwardly towards the pastern. The sleeve 1500 may be worn in a manner similar to a sock. Hence, in some implementations, the sleeve 1500 may surround the entirety of the pastern and extend to (and cover) the hoof. While shown as a sleeve 1500, in some implementations the sleeve 1500 may be adapted or modified as a strap, boots, etc.

The sleeve 1500 may be constructed of a flexible (or stretchy) material. For instance, the sleeve 1500 may be constructed from spandex, elastane, rayon, polyester, nylon, etc. and/or combinations of such materials. The sleeve 1500 may fit tightly around the fetlock. The sleeve 1500 may compress the fetlock (similar to compression materials, socks, etc.). In some instances, the sleeve 1500 may fit and be worn underneath the orthosis 10. In still some instances, the sleeve 1500 may be worn separately from the orthosis 10.

As described in greater detail below, the sleeve 1500 may include one or more sensors 1502, such as flexible capacitive fibers which may be woven into, sewn into, or otherwise incorporated into the sleeve 1500. The sensors 1502 may be used for measuring force(s) on the sleeve 1500. The sleeve 1500 may include a controller for identifying the force(s) based on measurements from the sensor(s) 1502, and a communication interface for communicating the detected force(s) to one or more external sources for analytics.

### B. Wearable Device for Monitoring Activity and Performance of an Equid

Various training and exercise regimens have been used to improve an equid's (for instance, horse's) performance. Typically, such training and exercise regimens are qualitatively analyzed and assessed to determine whether such training and exercise regimens are effective. Additionally, rehabilitation is also qualitatively assessed to determine effectiveness. However, such qualitative analysis does not provide any benchmarks for subsequent analysis. Rather, the horse's performance rests upon proper analysis of a trainer. Such analysis may be highly subjective and prone to error. In some instances, some trainers may not communicate their analysis to all parties, which may result in lack of communication, miscommunication, and error. Further, there is a general lack of quantitative data for horses.

Hence, it may be desirable to quantitatively analyze a horse's activity and performance. Further, it may be desirable to provide a communication system for providing such qualitative analysis to interested parties. By quantitatively analyzing a horse's activity and performance, fewer considerations are subjective.

The present disclosure is generally directed to systems and methods of recording and registering various measurements associated with equids, such as horses. Data may be generated which corresponds to the fetlock joint. For instance, the data may indicate forces acting on the fetlock joint, the cannon, and/or the pastern. The data may also indicate positions of each or one or more of the cannon or pastern, including relative positions of the cannon and pastern to each other (e.g., position data). In some instances, relative forces (e.g., within the same limb, acting on separate limbs, etc.) and positions may be used to track the activity of a horse or a performance of the horse. The data collected can then be used to identify various conditions of the horse, a baseline of the horse, monitor execution and performance relating to training regimens for the horse, monitor execution and performance relating to treatment regimens of the horse, monitor execution and performance relating to rehabilitation regimens of the horse, among others. In some embodiments, the relative forces acting on the one or more limbs of the horse may be used for identifying various metrics relating to the movement of the horse, including but not limited to determining a speed profile of the horse, a lead limb of the horse, any unusual or unique movements of the horse, missteps of the horse, among others. In some instances, such data may be used for performing a gait analysis for the horse. For instance, an owner, trainer, veterinarian, rider, jockey, or other entity may review the gait analysis generated by an equid performance analytics system in comparison to previous performances, performances of other horses, etc. Such comparison may be used for performing analytics. In some embodiments, the data may be used for determining a speed profile of the horse (e.g., how fast the horse is at counter, gallop, etc.).

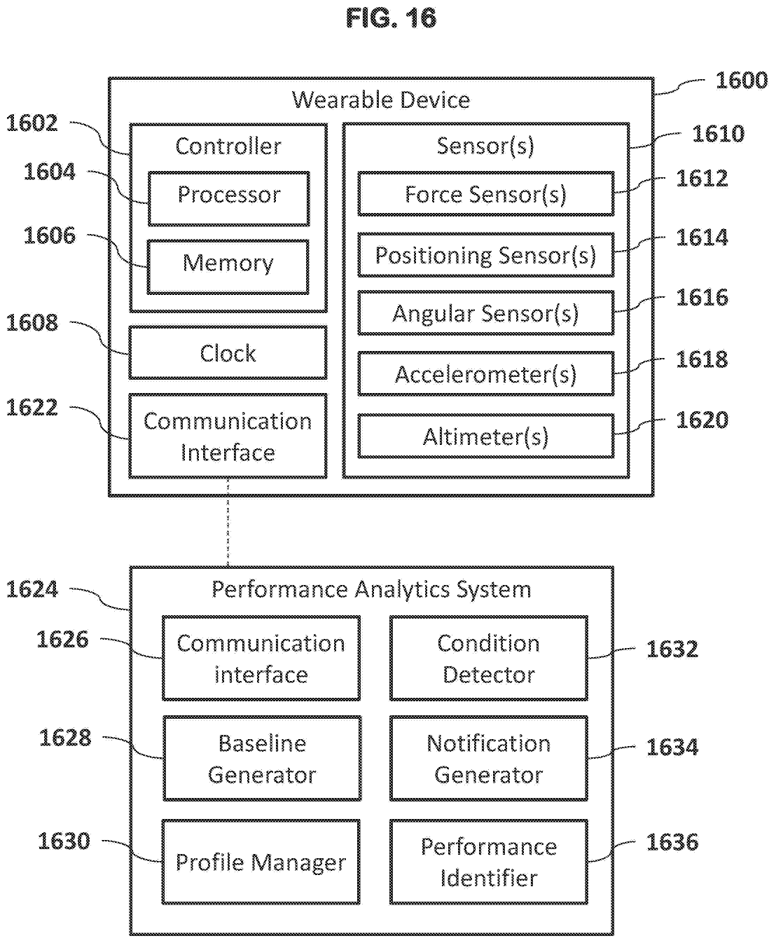

Referring now to FIG. 16, an analytics system including one or more wearable devices 1600 and a performance analytics system 1624 for collecting and analyzing data relating to activities of one or more horses is shown, according to an exemplary embodiment. In some embodiments, the wearable device 1600 may be embodied as the orthosis 10 described above. In some embodiments, the wearable device 1600 may be embodied as the sleeve 1500 described above. The wearable device 1600 may include a controller 1602 having a processor 1604 and memory 1606. The wearable device 1600 may include a clock 1610 for timestamping data generated by one or more sensors 1610. The wearable device 1600 may include or be communicably coupled to the sensor(s) 1610. The sensor(s) 1610 may be or include force sensor(s) 1612, positioning sensor(s) 1614, angular sensor(s) 1616, accelerometer(s) (or gyroscopes) 1618, and/or altimeter(s) 1620, among other types of sensors. The sensor(s) 1610 may generate data, and the wearable device 1600 may timestamp the data. The wearable device 1600 may include a communication interface 1622. The communication interface 1622 may communicate the data (which may or may not be processed by the processor(s) 1604) to a performance analytics system 1624 that can perform an analysis on the data and share the analysis with various interested parties, as described in greater detail below in section F.

The wearable device 1600 may include a controller 1602. While the controller 1602 is shown as included in the wearable device 1600, in some embodiments, the controller 1602 may be separate from but communicably coupled to the wearable device 1600. The controller 1602 may be or include a component or group of components configured to perform various functions for the wearable device 1600. For instance, the controller 1602 may include a processor 1604 and memory 1606. The processor 1604 may be a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. The processor 1604 also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function.

The memory 1606 (e.g., memory, memory unit, storage device) may include one or more devices (e.g., RAM, ROM, EPROM, EEPROM, optical disk storage, magnetic disk storage or other magnetic storage devices, flash memory, hard disk storage, or any other medium) for storing data and/or computer code for completing or facilitating the various processes, layers and modules described in the present disclosure. The memory 1606 may be or include volatile memory or non-volatile memory, and may include database components, object code components, script components, or any other type of information structure for supporting the various activities and information structures described in the present disclosure. According to an exemplary embodiment, the memory 1606 is communicably connected to the processor 1604 via a processing circuit and includes computer code for executing (e.g., by the processing circuit or the processor 1604) the one or more processes described herein.

In some embodiments, the wearable device 1600 may include a clock 1608. The clock 1608 may be a circuit or device configured to generate a signal which can be used for timestamping data. For instance, the clock 1608 may be a signal generator configured to generate a sinusoidal wave with predetermined or pre-known characteristics, such as frequency, period, pulse width, etc. In some instances, the clock 1608 may be an electronic oscillator regulated by a crystal, such as quartz. The clock 1608 may be communicably coupled to the sensor(s) 1610, the controller 1602, etc. The clock 1608 may generate a temporal signal which may be used by the sensor(s) 1609 and/or the controller 1602 for timestamping the sensor data described herein.

The wearable device 1600 may include one or more sensor(s) 1610. The sensor(s) 1610 may be a single sensor or a group of sensors. In instances where the sensor(s) 1610 are a group of sensors, the group of sensors may work together as a sensor array. The sensor(s) 1610 may be configured to detect and/or generate data corresponding to one or more conditions for the horse. For instance, the sensor(s) 1610 may be configured to detect forces acting on the fetlock joint, the cannon, the pastern, among other portions of the horse. In some embodiments, the sensor(s) 1610 may be configured to track the position (global or relative) of the leg. In some embodiments, the sensor(s) 1610 may be configured to track the acceleration (global or relative) of the leg. In some embodiments, the sensor(s) 1610 may be configured to track the angular rotation of the fetlock joint, the rotation of the horse's leg, etc. In some embodiments, the sensor(s) 1610 may be configured to track the height at which the horse jumps. The sensor(s) 1610 may be communicably coupled to the controller 1602. Hence, the sensor(s) 1610 may provide sensor data to the controller 1602 for processing. The sensor(s) 1610 may communicate the sensor data to the controller 1602 in real-time, near real-time, in intervals, etc. As described in greater detail below, the performance analytics system 1624 may use the sensor data for determining one or more conditions of the horse, for improving performance of the horse, for optimizing a training regimen of the horse, among others.

Where the wearable device 1600 is embodied as the orthosis 10, the sensor(s) 1610 may be mounted at various locations on or along the orthosis 10. For instance, the sensor(s) 1610 may be positioned on an interior surface of the orthosis 10. The sensor(s) 1610 may be located on, embedded within, or otherwise incorporated into the PU foam layer 20 and/or thermoformable foam layer 22 (of FIG. 2). Hence, the sensor(s) 1610 may have direct or indirect contact with the leg (e.g., the fetlock joint, the cannon, and/or the pastern). In some embodiments, the sensor(s) 1610 may be located along an exterior surface of the orthosis 10. For instance, the sensor(s) 1610 may contact features located along the exterior surface of the orthosis 10. The sensor(s) 1610 may thus measure indirect characteristics for the leg based on corresponding characteristics measured on the orthosis 10, as described in greater detail below. Where the wearable device 1600 is embodied as the sleeve 1500, in some embodiments, the sensor(s) 1610 may be located on or embedded within the sleeve 1500. The sleeve 1500 (and corresponding sensor(s) 1610) may conform to the leg. The sensor(s) 1610 in the sleeve 1500 may measure various characteristics for the leg, such as when the sleeve 1500 is worn underneath the orthosis 10 or separate from the orthosis 10.

In some embodiments, the sensor(s) 1610 may be arranged along the inner perimeter of the PU foam layer 20, thermoformed foam layer 22, and/or the sleeve 1500. The sensor(s) 1610 may be arranged relative to a center line (e.g., a line extending along a vertical axis separating the leg into a left side and right side). The sensor(s) 1610 may be arranged equidistant from the center line (e.g., along the cannon bone, along the pastern bone, etc.). Additionally, the sensor(s) 1610 may be arranged along the anterior, medial, and/or posterior portion of the leg to measure front, side, and back relative characteristics for the leg. In some embodiments, each leg may include a wearable device 1600 including sensor(s) 1610. Such sensor(s) 1610 may be used in conjunction to detect relative measurements, such as relative forces, position, acceleration, rotation, etc., as described in greater detail below.

In some embodiments, the sensor(s) 1610 may include force sensor(s) 1612. The force sensor(s) 1612 may be configured to measure, detect, identify, quantify, or otherwise generate data corresponding to a force and/or pressure acting on the sensor. For instance, the force sensor(s) 1612 may be or include piezoelectric sensor(s), strain gauges, etc. The force sensor(s) 1612 may be mounted at various locations along or within the wearable device 1600. The force sensor(s) 1612 may be designed or implemented to generate data corresponding to the force acting on the sensor, and correspondingly, on the fetlock joint, the cannon, and/or the pastern. The force sensor(s) 1612 may generate analog and/or digital data corresponding to the force acting on the sensor.

Referring now to FIG. 1 and FIG. 16, in some embodiments, the force sensor(s) 1612 may be mounted, attached or otherwise coupled to a surface on the stop 14b or stop 12b (of the orthosis 10). For instance, the force sensor(s) 1612 may be mounted, attached to, or otherwise coupled to the surface at the juncture between the stops 12b, 14b. The force sensor(s) 1612 may generate data corresponding to the force at which the stop 14b affixed to the distal cuff contacts the stop 12b affixed to the proximal cuff 12b. The stop 14b contacts the stop 12b during rotation of the fetlock joint. Hence, the force sensor(s) 1612 may generate data corresponding to the rotational force of the fetlock joint based on the force from the contact of the stop 14b to the stop 12b. In some embodiments, the rotational force of the fetlock joint may be translated (e.g., by the performance analytics system 1624 and/or the controller 1602) to a force from the ground exerted on (and through) the leg. For instance, where the stop 12b and/or stop 14b are located at a predetermined position (e.g., as described above, the relative position of one or the other of the stops can be varied to limit the ROM to a desired degree), the performance analytics system 1624 and/or controller 1602 may determine ROM angle. Hence, the force and angle may be used by the performance analytics system 1624 and/or controller 1602 for determining the force extending from the ground through the leg.

Where the wearable device 1600 is embodied as the orthosis 10, in some embodiments, the force sensor(s) 1612 may be mounted to or attached to the upper cuff 12 and/or lower cuff 14 of the orthosis 10. For instance, the force sensor(s) 1612 may be mounted or attached to an inner surface (e.g., longitudinally arranged along the inner surface) of the upper cuff 12 and/or lower cuff 14. The force sensor(s) 1612 may be embedded into PU foam layer 20 and/or the thermoformed foam layer 22 of the upper cuff 12 (and similar layer(s) for the lower cuff 14). The force sensor(s) 1612 may detect forces exerted from the cannon on the upper cuff 12 and the pastern on the lower cuff 14. In some embodiments, the upper cuff 12 (and/or lower cuff 14) may include multiple force sensor(s) 1612. For instance, the upper cuff 12 may include force sensors 1612 along the center line described above, equidistant distances from the center line, etc. Hence, the upper cuff 12 may include force sensor(s) 1612 around the cannon to detect forces on the left or right side of the cannon, posterior and anterior of the cannon, etc. Similarly, the lower cuff 14 may include force sensors 1612 along the center line described above, equidistant distances from the center line, etc. Thus, the lower cuff 14 may include force sensor(s) 1612 around the pastern to detect forces on the left or right side of the pastern, posterior and anterior of the pastern, etc. As the cannon or pastern pushes against the upper and lower cuff 12, 14, respectively, the cannon and pastern may exert a force on the upper and lower cuff 12, 14. The force sensor(s) 1612 may detect the force exerted on the upper and lower cuff 12, 14.

In embodiments where the wearable device 1600 is embodied as the sleeve 1500, in some embodiments, force sensor(s) 1612 may be embedded into or otherwise provided in the sleeve 1500. The force sensor(s) 1612 may be, for instance, conductor thread or other conductive fabric. The force sensor(s) 1612 may operate in a manner similar to smart fabrics. For instance, as the conductor thread flexes, a resistance (or inductance, capacitance, or other measurable electrical or electromagnetic property) pattern may be generated in proportion to the flex. The resistance pattern may be detected, registered, quantified, or otherwise identified by the controller 1602 to determine a force measurement. The force sensor(s) 1612 may be provided in the sleeve 1500 at various locations. For instance, the force sensor(s) 1612 may be arranged or otherwise situated or located near the cannon, the pastern, the fetlock joint, along the center line, equidistant from the centerline, etc. The force sensor(s) 1612 may thus generate data corresponding to forces exerted by the fetlock joint, the cannon, and/or the pastern, relative sides and portions of the fetlock joint, the cannon, and/or the pastern, etc.

In some embodiments, the sensor(s) 1610 may include positioning sensor(s) 1614. The positioning sensor(s) 1614 may be configured to measure, detect, identify, quantify, or otherwise generate data corresponding to a position or location of the sensor. In some embodiments, the positioning sensor(s) 1614 may detect relative positions and/or global (or absolute) positions. For instance, one positioning sensor 1614 may detect a relative position (or displacement) with respect to one or more other positioning sensor(s) 1614. In some embodiments, the positioning sensor(s) 1614 may detect global positions (e.g., the positioning sensor(s) 1614 may be similar to GPS).

In some embodiments, the positioning sensor(s) 1614 may be provided in, incorporated into, embedded into, included in, or otherwise coupled to the wearable device 1600. For instance, the positioning sensor(s) 1614 may be arranged or included along an exterior surface (e.g., outer surface) of the upper or lower cuff 12, 14 of the orthosis 10, the sleeve 1500, etc.

The positioning sensor(s) 1614 may be communicably coupled to the controller 1602. The positioning sensor(s) 1614 may provide sensor data corresponding to detected relative or global positions of the sensor 1614 to the controller 1602 for processing. The positioning sensor(s) 1614 may generate positional sensor data, which may be used by the performance analytics system 1624 for determining, for instance, a speed profile of the horse, lead leg of the horse, steps taken by the horse, missteps, etc. The speed profile can identify a top speed of the horse, an average speed, as well as different speeds corresponding to a cantor, gallop, among others. Additionally, the performance analytics system 1624 may use the positional sensor data for determining agility of the horse. For instance, as the horse moves back and forth (such as in dressage or other performance), the positioning sensor(s) 1614 may generate data corresponding to the horse's performance based on the detected position of the positioning sensor(s) 1614. The performance analytics system 1624 may use the positional sensor data for tracking a location or path of the horse. In some embodiments, the positioning sensor(s) 1614 can include or use data from one or more other types of sensors, including angular sensor(s) 1616, such as gyroscopes, as well as accelerometer(s) 1618 and altimeter(s) 1620, among others.

In some embodiments, the sensor(s) 1610 may include angular sensor(s) 1616. The angular sensor(s) 1616 may be configured to measure, detect, identify, quantify, or otherwise generate data corresponding to an angular rotation. In some embodiments, the angular sensor(s) 1616 may be or include rotary sensors or encoders, hall effect sensors, etc. The angular sensor(s) 1616 may be provided in or otherwise incorporated into or coupled to the pivot structure 24 of the orthosis 10. For instance, as the pivot structure 24 rotates, the angular sensor(s) 1616 may generate data corresponding to the rotation. The angular sensor(s) 1616 may generate angular sensor data corresponding to the rotation of the pivot structure 24.

The angular sensor(s) 1616 may be communicably coupled to the controller 1602. The angular sensor(s) 1616 may provide sensor data corresponding to detected rotation of the sensor 1616 to the controller 1602 for processing. The angular sensor(s) 1616 may generate rotational sensor data, which may be used by the controller 1602 and/or performance analytics system 1624 for determining, for instance, fetlock rotation or angle, change in fetlock rotation or angle over time (e.g., during a race from start to finish, over time of treatment or training, etc.), hyperextension, and other analysis.

In some embodiments, the angular sensor(s) 1616 can include gyroscope(s). The gyroscope(s) may be e mounted, attached to, or otherwise included within or coupled to the wearable device 1600. For instance, the gyroscope(s) may be included in or along the inner or outer surface or embedded within the orthosis 10 (similar to the force sensor(s) 1612 described above) or the sleeve 1500. The gyroscope(s) may generate rotation data corresponding to the body on which the gyroscope(s) are mounted. The gyroscope(s) may be communicably coupled to and provide the rotation data to the controller 1602 and/or performance analytics system 1624 for processing.

In some embodiments, the sensor(s) 1610 may include accelerometer(s) 1618. The accelerometer(s) 1618 may be configured to measure, detect, identify, quantify, or otherwise generate data corresponding to accelerations for the sensor. The accelerometer(s) 1618 may detect accelerations in three axes (e.g., the accelerometer 1618 may be a three-axis accelerometer). In some embodiments, the accelerometer(s) 1618 may detect relative acceleration of the front versus hind legs. For instance, the accelerometer(s) 1618 may detect relative acceleration for identifying missteps or bucks, sliding or slipping, etc. In some embodiments, the rotation data from the gyroscope(s) and the acceleration data from the accelerometer(s) 1618 can be used to determine a given position and orientation of one or more components of the wearable device 1600.

The accelerometer(s) 1618 may be mounted, attached to, or otherwise included within or coupled to the wearable device 1600. For instance, the accelerometer(s) 1618 may be included in or along the inner or outer surface or embedded within the orthosis 10 (similar to the force sensor(s) 1612 described above) or the sleeve 1500. The accelerometer(s) 1618 may generate acceleration data corresponding to the body on which the accelerometer(s) 1618 are mounted. The accelerometer(s) 1618 may be communicably coupled to and provide the acceleration data to the controller 1602 and/or performance analytics system 1624 for processing.

In some embodiments, the sensor(s) 1610 may include altimeter(s) 1620. The altimeter(s) 1620 may be configured to measure, detect, identify, quantify, or otherwise generate data corresponding to elevation. The altimeter(s) 1620 may measure relative global (or absolute) elevation. The altimeter(s) 1620 may measure relative elevation (e.g., elevation of one altimeter 1620 with respect to one or more other altimeter(s) 1620). The altimeter(s) 1620 may measure how high a horse is jumping. In some embodiments, the altimeter(s) 1620 may work together with one or more other sensor(s) 1610, such as force sensor(s) 1612. The altimeter(s) 1620 together with the force sensor(s) 1612 may measure how high a horse jumps, and resulting force at impact (or leading up to the jump) at one or more locations on the leg or body of the horse.

The altimeter(s) 1620 may be mounted, attached, provided in or within or otherwise coupled to the wearable device 1600. The altimeter(s) 1620 may generate elevation sensor data, which may be provided to the controller 1602 for processing.

While various examples of sensor(s) 1610 are described herein, the present disclosure contemplates any number of sensor(s) 1610 which may provide sensor data that may assist in diagnosing conditions for a horse, evaluating recovery or training, etc., as described in greater detail below. In some embodiments, the sensor(s) 1610 may be used by the performance analytics system 1624 for evaluating a duration that a horse has ran (e.g., training time or duration), number of steps or strides taken, analysis or changes of a horse's stride, jump tracking and analysis (e.g., jump height, jump angles, etc.), distance traveled, maximum speed, gait analysis, force analysis on various portions of the leg or body of the horse, identifying missteps or bucks, diagnosing conditions such as colic or agitation, determining lead leg or changes in lead leg, as described in greater detail below.

The performance analytics system 1624 may be or include a device or component (or group of devices or components) configured or designed to process data from one or more wearable devices, such as the wearable device 1600. In some embodiments, the wearable devices may be configured to communicate with a computing device, such as a smartphone, tablet, laptop, or any other computing device that can further communicate with the performance analytics system 1624. In some embodiments the computing device can include an application configured to cause the computing device to communicate with and transmit and receive data to and from the performance analytics system 1624. In some embodiments, the wearable devices can be associated with the same horse. In some embodiments, multiple wearable devices collecting data from a plurality of horses can be shared with the performance analytics system 1624. In some embodiments, the performance analytics system 1624 may include various processors, memory, controllers, etc., similar in some aspects to those described above with reference to the wearable device 1600. The performance analytics system 1624 may quantitatively assess a horse based on sensor data generated from the horse while the horse wears the one or more wearable devices 1600. The performance analytics system 1624 may compare sensor data from the wearable devices 1600 to baseline data. The performance analytics system 1624 may determine, evaluate, or otherwise identify one or more characteristics or conditions for a horse based on the analysis. The performance analytics system 1624 may disburse such characteristics or conditions to interested parties, such as the owner of the horse, the veterinarian, the jockey or trainers, etc.

C. Systems and Methods for Generating one or more Baselines for a Horse

In some embodiments, the performance analytics system 1624 may create, form, identify, or otherwise generate one or more baseline measurements. “Baseline,” as used herein, refers to a dataset used for comparison. Hence, the baseline measurements may correspond to a horse which is used by the performance analytics system 1624 for comparison to other horses or the same horse at a different point in time (e.g., following injury, during recovery, during training). In some embodiments, the data used by the performance analytics system 1624 for generating the baseline may be recorded or otherwise stored by the controller 1602 of the wearable device 1600 (e.g., by selecting a baseline button or input device prior to training or exercising the horse), or separate from the controller 1602 (e.g., by a separate computer or portal that receives or otherwise downloads the data from the wearable device 1600).

The performance analytics system 1624 may include a communication interface 1626. The communication interface 1626 may be communicably coupled to the communication interface 1624 for the wearable device 1600. In some embodiments, the communication interface 1626 for the performance analytics system 1624 may be communicably coupled to the wearable device 1600 via a computing device configured to communicate with one or more wearable devices 1600 and the performance analytics system 1624. The communication interfaces 1626, 1622 may be communicably coupled to one another via a computer network. The computer network may be a Local Area Network (LAN), a Wide Area Network (WAN), a Wireless Local Area Network (WLAN), an Internet Area Network (IAN), a cloud-based network, etc. In some implementations, the communication interface 1622 may access the computer network to exchange data with the communications device 1626 via cellular access, a modem, broadband, Wi-Fi, Bluetooth, satellite access, etc. The communication interface 1622 may provide data to the performance analytic system 1624 (e.g., via the communication interface 1626) upon request, at intervals, and/or in real-time. In some embodiments, the performance analytics system 1624 may request an update by communicating a signal via the communication interface 1626 to the communication interface 1622 for the wearable device 1622. The communication interface 1622 may provide the update with the corresponding sensor data to the performance analytics system 1624. Hence, the wearable device 1600 may exchange data with the performance analytics system 1624 via their respective communication interfaces 1622, 1626 (and any intervening computing devices).

In some embodiments, the performance analytics system 1624 may receive data from the wearable device 1600 for generating a baseline. For instance, the performance analytics system 1624 may include a baseline generator 1628. The baseline generator 1628 may be any device, component, or group of devices or components configured to or designed to generate a baseline from sensor data. In some embodiments, the baseline generator 1628 may be embodied as a dedicated processor and memory, where the memory stores instructions for the processor to generate the baseline. The baseline generator 1628 may be embodied as instructions stored on memory executable by a separate processor for the performance analytic system 1624.

In some embodiments, the performance analytics system 1624 may include or access data corresponding to movement of a healthy horse. The movement may correspond to the horse walking, trotting, jumping, etc. In one or more implementations, the data may be or include motion data, such as rotation and translation data. The rotation data may be or include rotation along the x-axis (e.g., flexion [FL] and extension [EX]), the y (or floating)-axis (e.g., abduction [AB] and adduction [AD]), and z-axis (e.g., external [EX] and internal [IN]). The x-axis may be defined by the third metacarpal bone (MCIII), the z-axis may be defined by the proximal phalanx (PI), and the y-axis may be defined as remaining perpendicular to both the x and z-axes. Translations may be defined by lateral (LA) and medial (ME) translations (left-right), cranial (CR) and caudal (CA) translations (up-down), and proximal (PR) and distal (DI) translations (front-back). The rotational and translational motion may be with respect to a center of gravity of the horse, a center of the fetlock joint, etc. The data may be ranges of data corresponding to such motion. An example of such data is shown in Table 1 below.

TABLE 1
Fetlock Joint Range of Motion

| | Rotations (°) [approx.] | | | Translations (mm) [approx . . . ] | | |
|---|---|---|---|---|---|---|
| | FL/EX | AB/AD | EX/IN | LA/ME | CR/CA | PR/DI |
| Walk | 25-30 | 0.2-1.0 | 0.0-0.6 | −0.1-1.1 | 0.5-1.0 | 0.1-0.6 |
| Trot | 29-37 | −0.2-0.8 | −0.5-1.8 | −0.15-1.2 | 0.8-1.3 | 0.3-0.9 |
| Jump | 25-55 | 0.9-2.0 | 0.5-5.5 | 0.1-0.5 | 0.6-0.9 | 0.5-0.8 |

Such data described above in Table 1 may be used for calculating or otherwise generating a baseline for a horse.

The performance analytics system 1624 may use the baseline or comparing the horse to one or more other horses or to the same horse at different times. The performance analytics system 1624 may compare data from the one or more wearable devices 1600 worn by the horse to a baseline for evaluating conditions of the horse, for determining optimal training regimen, determining progress in training or recovery, as described in greater detail below. The baseline may include force or pressure data (e.g., generated by force sensor(s) 1612), gait analysis data (e.g., generated by force and positioning sensor(s) 1612, 1614), fetlock rotation data (e.g., generated by angular sensor(s) 1616, positioning sensor(s) 1614, and/or force sensor(s) 1612), jumping height data (e.g., generated by altimeter(s) 1620, accelerometer(s) 1620, positioning sensor(s) 1614, and/or force sensor(s) 1612), agility data (e.g., generated by positioning sensor(s) 1614, accelerometer(s) 1618), speed data (e.g., generated by positioning sensor(s) 1614 and/or accelerometer(s) 1618), and other baseline data that may be used for comparison. In some embodiments, the baseline may include individual data corresponding to a single leg of the horse, and the baseline may include relative data corresponding to comparative data for each leg of the horse. In some embodiments, the individual data corresponding to a single leg may itself include comparative data corresponding to different sides of the leg, anterior versus posterior data, etc.

In some embodiments, the baseline generator 1628 may generate the baseline based on data from a healthy horse. The baseline generator 1628 may generate the baseline following a trainer or veterinarian providing the orthosis 10 or sleeve 1500 on the healthy horse and, for instance, selecting an option on the wearable device 1600 or a computing device coupled to the wearable device 1600 for recording one or more sensor measurements. For instance, the healthy horse may run, trot, gallop, jump, etc., and the sensor(s) 1610 may generate sensor data for the horse. The sensor data may be compiled by the controller 1602 and communicated to the performance analytics system 1624 (e.g., via the respective communication interfaces 1622, 1626). The baseline generator 1628 may generate the baseline based on or corresponding to the sensor data compiled by the controller 1602. In some embodiments, the baseline generator 1628 may average compiled sensor data over time to generate the baseline.

In some embodiments, the baseline may be a baseline for a specific horse for comparison to data generated by the same horse at a different point in time. The performance analytics system 1624 may include a profile manager 1630. The profile manager 1630 may be or include any device or component (or group of devices or components) configured to generate and manage a profile associated with a horse. The profile manager 1630 may be embodied as a dedicated processor and memory, or instructions stored on memory executable by a separate processor for the performance analytic system 1624. The profile manager 1630 may receive or otherwise generate an identifier for a horse (e.g., from an owner, trainer, veterinarian, etc.) which is uniquely associated with the horse. The profile manager 1630 may store the identifier in a profile for the horse. The wearable device 1600 may be paired or otherwise associated with the horse by providing an identifier to the performance analytics system 1624. The profile manager 1630 may associate the wearable device 1600 with the profile for the horse. In some embodiments, the profile manager 1630 may associate multiple wearable devices 1600 with a horse (e.g., the wearable device for each leg, for instance). Hence, the profile for a given horse may be associated with a number of wearable devices 1600.

For instance, a veterinarian or trainer may fit the wearable device 1600 to the horse at a point in time when the horse is deemed healthy. The horse may be exercised, and the sensor(s) 1610 provided in the wearable device 1600 (e.g., the orthosis 10/sleeve 1500) may generate sensor data corresponding to the horse's exercise. The data may be compiled by the controller 1602, and sent via the communication interface 1622 to the communication interface 1626 for the performance analytics system 1624. The baseline generator 1628 may use the received and compiled sensor data to generate a baseline. The baseline generator 1628 may provide the baseline to the profile manager 1630 for inclusion, incorporation, or otherwise association with a profile for a particular horse. As the baseline generator 1628 modifies the baseline for a particular horse, the profile manager 1630 may correspondingly update the baseline associated with the horse in the horse's profile.

Subsequently, the horse may be trained, further exercised, etc. while wearing the wearable device 1600, and the wearable device 1600 may communicate the data generated at that point in time to the performance analytics system 1624. The performance analytics system 1624 may compare the received data to the baseline data for that particular horse. In some embodiments, the performance analytics system 1624 may evaluate the comparison to determine whether the training and exercising regimen is suitable for the horse. Hence, the performance analytics system 1624 may dynamically update training regimens or rehabilitation/treatment plans for a horse based on performance or other sensor data received from the wearable device 1600. In some embodiments, trainers, jockeys, owners, veterinarians, etc., may evaluate the comparison to determine whether the training and exercising is suitable for the horse. In each of these embodiments, various aspects of a horse's regimen may be modified following analysis of the data from the wearable device 1600, as described in greater detail below. Hence, the baseline may be a baseline for a specific horse, and the baseline may be used for comparison of data for the horse at subsequent points in time.

In some embodiments, the baseline may be a baseline for a group of horses. For instance, the baseline may be specific to a group of similarly situated horses. Horses may be grouped by the baseline generator 1628 according to various different characteristics. Horses may be grouped by the baseline generator 1628 based on, for instance, breed, age, discipline, condition, etc. Such data may be provided, included, or otherwise incorporated into the profile maintained by the profile manager 1630. The profile manager 1630 may receive (e.g., from an owner, trainer, veterinarian, etc.) various characteristics of a horse, such as breed, age, discipline, conditions, etc. The profile manager 1630 may include such characteristics with the profile for the horse. The baseline generator 1628 may sort or otherwise access profiles having a particular characteristics for forming a baseline for that particular characteristic.

As one example, horses of the same breed may include a baseline for their specific breed. As another example, horses of the same discipline (e.g., rodeo horses, eventing horses, hunter/jumper horses, racing horses, polo horses, etc.) may have a baseline for their specific discipline. As still another example, horses having the same condition (e.g., lameness, colic, bowed tendons, etc.) may have a baseline for their specific condition, which may be used by the performance analytics system 1624 for diagnosing such conditions, and detecting improvements from such conditions. As yet another example, horses may be grouped based on the condition in which they are exercising, running, training, etc. For instance, where the conditions of a track are muddy, a baseline may be generated for horses on muddy tracks. In some embodiments, a baseline may be formed for sub-groups (e.g., a breed of horses on a muddy track, race horses having lameness, two-year-old polo horses, etc.). Hence, the baseline may have different layers of granularity such that similarly situated horses may be compared and evaluated against a granular baseline.

As described in greater detail below, a given horse may be compared by the performance analytics system 1624 to baseline data corresponding to the horse. The performance analytics system 1624 may detect or identify a deviation from the baseline (e.g., improvements or digressions). The comparison may be used by the performance analytics system 1624 for identifying improvements in conditioning or training of the horse, for identifying potential changes in training, for early diagnosis or prediction of conditions of the horse, for predicting performance of a horse over time, for predicting effective training or rehabilitation programs for a horse, for predicting a timeline for completing rehabilitation, etc.

### D. Performance Analytics System for Analytically Detecting Conditions for the Horse

The performance analytics system 1624 may use sensor data generated by the sensor(s) 1610 for evaluating the horse. For instance, the sensor data generated by the sensor(s) 1610 may be compared (e.g., by the performance analytics system 1624) to the baseline described above in section C. In some embodiments, the sensor data may be informative even without comparison. Hence, the performance analytics system 1624 may detect or identify some conditions without comparing sensor data to a baseline.

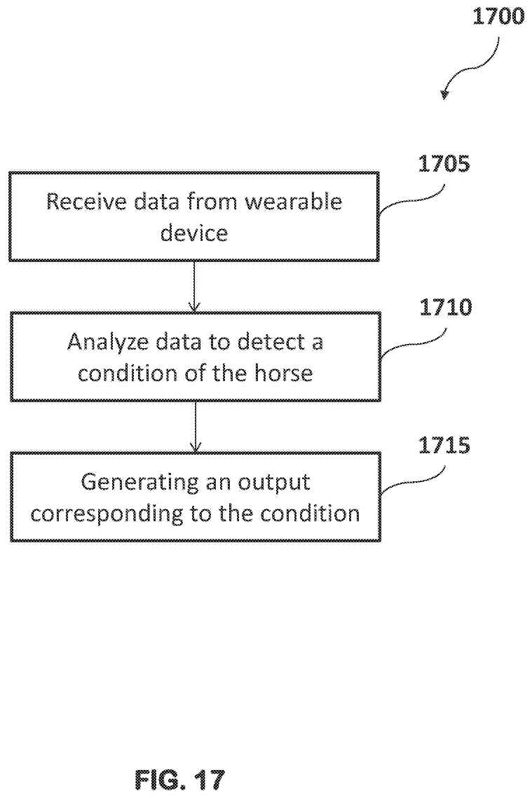

Referring now to FIG. 17, a flowchart showing an example method 1700 for analyzing sensor data corresponding to a leg of a horse is shown, according to an exemplary embodiment. The method 1700 is shown to include receiving data from a wearable device (operation 1705), analyzing the data to detect a condition of the horse (operation 1710), and generating an output corresponding to the condition (operation 1715).

At operation 1705, a performance analytics system 1624 may receive data from a wearable device 1600. In some embodiments, the performance analytics system 1624 may receive the data from the wearable device 1600 via respective communication interfaces 1622, 1626. The wearable device 1600 may be the orthosis 10 and/or the sleeve 1500. The orthosis 10 and/or sleeve 1500 may include one or more sensor(s) 1610 which generate sensor data corresponding to a leg of the horse and a controller 1602 communicably coupled to the one or more sensor(s) 1610. The wearable device 1600 may generate the sensor data corresponding to a leg of a horse. The sensor data may be force sensor data, position sensor data, angular sensor data, acceleration sensor data, and altitude sensor data. The controller 1602 may control a communication interface 1622 to communicate the sensor data to a communication interface 1626 for a performance analytics system 1626.

At operation 1710, the performance analytics system 1624 may analyze the sensor data to detect a condition of the horse. Several conditions and evaluations are described herein. However, the present disclosure is not limited to these particular evaluations and conditions. Rather, the present disclosure provides examples of evaluating a horse based on sensor data generated from a wearable device 1600 provided on one or more legs of a horse. The performance analytics system 1624 may receive the sensor data from the one or more sensor(s) 1610. In some embodiments, the performance analytics system 1624 may compare the sensor data to a baseline for a similarly situated horse. The performance analytics system 1624 may determine, evaluate, or otherwise identify one or more conditions of the horse based on the sensor data from the one or more sensor(s) 1610.

Following analyzing the data to detect the condition of the horse, the method 1700 may include generating an output 1710 corresponding to the condition. As described in greater detail below, the output may depend on the condition. The output may include, for instance, a notification, a modification to training, rehabilitation, exercise routine or regimen for the horse, etc.

In some embodiments, the sensor data may be used for detecting a condition. Such a condition may be or include agitations, ailments, or other injuries, including, for instance, colic, fetlock strain, flexor strain, etc. The performance analytics system 1624 may include a condition detector 1632. The condition detector 1632 may be any device, component or group of devices or components configured to designed to detect one or more conditions for a horse based on sensor data. The condition detector 1632 may be embodied as a dedicated processor and memory, or instructions stored on memory executable by a separate processor for the performance analytic system 1624. The condition detector 1632 may include or use condition data. The condition data may be or include data which is associated with, indicates, or otherwise suggests a horse has a particular condition. The condition detector 1632 may use the condition data for identifying corresponding conditions with a horse.

The method 1700 may include receiving, from one or more sensor(s) 1610, sensor data for a wearable device 1600. The sensor data may be received in real-time, near real-time, or at intervals. The sensor data may be generated by the sensor(s) 1610 while the horse is exercising or training, while the horse is walking, etc. The sensor data may be received by the controller 1602 of the wearable device 1600. The controller 1602 may package, process, or otherwise compile the sensor data, and may communicate the sensor data to the performance analytics system 1624. The condition detector 1632 may analyze the sensor data. The condition detector 1632 may use the condition data for comparing to the sensor data. The condition detector 1632 may identify or otherwise flag one or more conditions in the sensor data based on the condition data. In some embodiments, the performance analytics system 1624 may generate (e.g., via a notification generator 1634 described in greater detail below) a notification to communicate to one or more portals associate with an owner, veterinarian, trainer, etc., which indicates the detected condition. Some of those conditions and example sensor data which may indicate such conditions are described herein.

In some embodiments, the condition may include horse fatigue. The horse may be over-exercised or over-trained in some instances. Such instances may cause the horse to become fatigued. Some of the sensor(s) 1610 for the wearable device 1600 may generate sensor data which may indicate the horse has become fatigued. The sensor data may be communicated from the wearable device 1600 to the performance analytics system 1624. The condition detector 1632 may analyze the sensor data from the wearable device 1600 to detect fatigue for the horse. The condition detector 1632 may include condition data corresponding to a fatigue condition.

As one example, horse fatigue may be detected based on hyper-extended forelimbs. As the horse exercises or trains, the horse may begin to hyper-extend their forelimb as the horse becomes fatigued, which may cause increased force on the fetlock joint and/or over-rotation (e.g., hyperextension) of the fetlock joint. Such a condition may be detected via the force sensor(s) 1612. The force sensor(s) 1612 may generate data showing increased force on the fetlock joint from the beginning of training or exercise. Such a condition may also be detected via the angular sensor(s) 1616. The angular sensor(s) 1616 may generate data showing an increased angle of extension of the fetlock joint from the beginning of training or exercise to the end of training or exercise. Such a condition may also be generated via the positioning sensor(s) 1614 and/or altimeters. The positioning sensor(s) 1614 and/or altimeters may show the horse is dipping its forelimbs over time from the beginning of training or exercise to the end. In each of these examples, the sensor(s) 1610 may generate data for the horse over a training or exercise session. The sensor data generated by the sensor(s) 1610 may show the horse is dipping its forelimb towards the end of the training or exercise session. The wearable device 1600 may communicate such sensor data to the performance analytics system 1624, where the condition detector 1632 analyzes such sensor data. The condition detector 1632 may determine the horse is fatigued based on such sensor data. In some embodiments, when the condition detector 1632 detects a condition, the notification generator 1634 may generate a corresponding notification. The notification may indicate the condition was diagnosed or otherwise identified or detected by the condition detector 1632. The performance analytics system 1624 may communicate the notification via the communication interface 1626 to one of the portals (e.g., the trainer portal, owner portal, veterinarian portal, etc.) described below with reference to FIG. 17.

In some embodiments, when the horse is fatigued or otherwise having an increased fetlock angle over the course of a training session, competition, or race, the increased fetlock angle may be compared to a threshold. When the fetlock angle for the horse exceeds a threshold, the notification generator 1634 may generate a notification which may be communicated via the communication interface 1622 to the veterinarian, the owner, the trainer, the jockey, etc. The notification may show the increased in fetlock angle (e.g., that the fetlock drop has exceeded a threshold). The owner/trainer may recommend treatment, modifying training, pulling the horse from further competition or subsequent races, etc.

In some embodiments, when the horse is fatigued, the horse may experience high-impact loading (especially in asymmetric limb loading). Such high-impact loading may be a damaging aspect in exercise. The sensors described above may be designed or implemented to detect and generate data corresponding to such high-impact loading. Additionally, as described herein, relative data with respect to different limbs of the horse may be used for detecting asymmetric limb loading. Such types of loading may increase the likelihood of subclinical tendon damage. The notification generator 1634 may generate a notification indicating high-impact loading and/or asymmetric limb loading. The owner/trainer may recommend treatment, modifying training, pulling the horse from further competition and/or subsequent races, etc.

In some embodiments, when the horse is fatigued, the horse may have an increased heart rate. In some embodiments, the sensor(s) 1610 may include a heart rate monitor. The heart rate monitor may be configured or designed to contact the horse for measuring the horse's heart rate. In some embodiments, the heart rate monitor may be a third-party heart rate monitor communicably coupled to the performance analytics system 1624 (e.g., associated with a profile for the horse via the profile manager 1630). The heart rate monitor may report the heart rate to the performance analytics system 1624, and the condition detector 1632 may determine, based on the heart rate of the horse, whether the horse is fatigued (e.g., the heart rate increasing at a rate exceeding a threshold, the heart rate itself exceeding a threshold, etc.). The notification generator 1634 may generate a notification which indicates the increased heart rate and/or the horse being fatigued.

In some embodiments, the condition may include lameness. Lameness may be caused by injury to one or more legs of the horse. Some of the sensor(s) 1610 for the wearable device 1600 may generate sensor data which may indicate the horse is experiencing lameness. Lameness may be manifested by the horse changing its gait.

In some instances, lameness may be detected by a change in forces on the lame leg. For instance, where the one or more of the front limbs are lame, the horse may bob its head (e.g., lift or raise the horse's head prior to the lame limb hitting the ground). The horse may bob its head to reduce the force on the lame limb. Similarly, the horse may hike their hips or pelvis to reduce the force on the lame rear limb prior to the lame rear limb hitting the ground. Such a condition may be detected via the force sensor(s) 1612. The force sensor(s) 1612 may generate data showing decreased force on the lame leg as the leg hits the ground. The force sensor(s) 1612 may register decreased forces at the fetlock joint, at the cannon or pastern, etc. of the lame limb.

In some instances, lameness may be detected based on the relative time a leg spends in the cranial (forward) phase versus the caudal (reverse) phase of a stride. For instance, a healthy horse may spend substantially the same time in the cranial phase and caudal phase of a stride. A lame horse may have a cranial phase that is shorter than the caudal phase of a stride. Such a condition may be detected by the accelerometer(s) 1618 and/or the positioning sensor(s) 1614. For instance, the accelerometer(s) 1618 may register a change in acceleration corresponding to the shift from the cranial phase to the caudal phase. Similarly the positioning sensor(s) 1614 may track the position of the leg as it transitions from the cranial to the caudal phase of a stride. The controller 1602 may determine the time elapsed in the cranial versus the caudal phase (e.g., via the transition time and using the clock 1608). The controller 1602 compare the time elapsed in the cranial phase to the time in the caudal phase. The controller 1602 may determine that a limb is lame when the time elapsed in the cranial phase exceeds the time elapsed in the caudal phase (e.g., by a nominal duration, for instance).

In some instances, lameness may be detected based on a decreased rotation of the fetlock joint. A horse may decrease the fetlock drop (e.g., the rotation of the fetlock joint) during a stance phase of the stride in comparison to a healthy leg. Hence, one leg may be more upright than another leg. The more upright leg may be the lame leg. The horse may decrease fetlock drop to relieve weight on the painful limb. Such a condition may also be detected via the angular sensor(s) 1616. The angular sensor(s) 1616 may generate data showing an decreased angle of extension of the fetlock joint. Such a condition may also be generated via the positioning sensor(s) 1614 and/or altimeters. The positioning sensor(s) 1614 and/or altimeters may show the fetlock drop of one limb is less than other limbs.

In each of these instances, the horse, in attempting to relieve pain, may change their gait. Such a change may be detected in a number of ways for diagnosing lameness. The controller 1602 may identify the lame leg to a trainer, owner, veterinarian, etc. for verification of lameness, treatment, training change, etc.

The wearable device 1600 may communicate such sensor data to the performance analytics system 1624, where the condition detector 1632 analyzes such sensor data. The condition detector 1632 may determine one of the horse's limbs are lame based on such sensor data. In some embodiments, when the condition detector 1632 detects a condition, the notification generator 1634 may generate a corresponding notification. The notification may indicate the condition diagnosed or otherwise identified or detected by the condition detector 1632. The performance analytics system 1624 may communicate the notification via the communication interface 1626 to one of the portals (e.g., the trainer portal, owner portal, veterinarian portal, etc.) described below with reference to FIG. 17.

In some embodiments, the condition may include colic. Colic may be a clinical sign of abdominal pain caused by a gastrointestinal condition, for instance. Colic may be manifested in a number of different ways. The colic may be detected by abnormal movements of the limbs at night. For instance, when a horse experiencing colic is attempting to sleep, the horse may successively move between a supine position to a standing position or may roll over at night. Such successive moving and rolling over may be detected via any of the above-mentioned sensor(s) 1610, which generally register data which may indicate the horse has moved from a supine to a standing position (e.g., increased elevation, forces on the legs/joints resulting from standing, extending or otherwise rotating the fetlock joint to stand, etc.). The wearable device 1600 may communicate such sensor data to the performance analytics system 1624, where the condition detector 1632 analyzes such sensor data. The condition detector 1632 may determine the horse is experiencing colic based on such sensor data. In some embodiments, when the condition detector 1632 detects a condition, the notification generator 1634 may generate a corresponding notification. The notification may indicate the condition diagnosed or otherwise identified or detected by the condition detector 1632. The performance analytics system 1624 may communicate the notification via the communication interface 1626 to one of the portals (e.g., the trainer portal, owner portal, veterinarian portal, etc.) described below with reference to FIG. 17. For instance, where the horse successively moves between the supine and standing position or rolls over at night, the notification generator 1634 may generate a notification which indicates the horse is experiencing colic. The notification may be communicated to a veterinarian, a trainer, an owner, etc., who may evaluate the horse for identifying the condition that may be causing the colic.

In some embodiments, the sensor data may be used for tracking the performance of a horse. The performance analytics system 1624 may include a performance identifier 1636. The performance identifier 1636 may be any device, component or group of devices or components configured to designed to detect, identify, or otherwise evaluate a horse's performance based on sensor data. The performance identifier 1636 may be embodied as a dedicated processor and memory, or instructions stored on memory executable by a separate processor for the performance analytics system 1624.

The wearable device 1600 may be worn on each or a subset of legs for the horse. The wearable device 1600 may be embodied as the orthosis 10, or the wearable device 1600 may be embodied as the sleeve 1500. In embodiments where the wearable device 1600 is embodied as the orthosis 10, the orthosis 10 may be constructed or a more light-weight material to lessen the weight on the leg of the horse. For instance, various metal parts may be forgone and replaced with light-weight materials, such as plastics.

The wearable device 1600 may be worn while the horse trains or exercises. The sensor(s) 1610 may generate data for the horse as the horse trains/exercises. The sensor data may be communicated (e.g., via the communication interfaces 1622, 1626) from the wearable device 1600 to the performance analytics system 1624. The performance identifier 1636 may plot the sensor data over time (e.g., in subsequent training or exercise sessions). The data may include speed, agility data, jump height data, etc., which may be collected by the positioning sensor(s) 1614, the accelerometer(s) 1618, the altimeter(s) 1620, etc. The performance identifier 1636 may identify trends in the plotted data. The plotted data over time may show the horse has improved or decreased performance. Such information may be used for decreasing rehabilitation time, for improving treatment protocols (e.g., modalities), for improving training regimens, etc. as described in greater detail below. For instance, the performance analytics system 1624 may dynamically adjust treatment protocols or modalities, training regimens, etc., based on such trends.

In some embodiments, the performance identifier 1636 may be configured to infer various information from the sensor data received from the wearable device 1600. For instance, the performance identifier 1636 may be configured to receive the speed, distance, forces, jump height data, etc. from the sensors of the wearable device 1600. The performance identifier 1636 may be configured to compute, estimate, or otherwise determine various information corresponding to the horse from such sensor data. As one example, the performance identifier 1636 may be configured to determine a number of calories burned by the horse during training using a duration of training, speed of the horse, distance traveled, forces, jumps, etc. Such information may be used for generating notifications corresponding to feeding the horse.

In some embodiments, the horse may have previously been injured and is undergoing rehabilitation or treatment. The horse may be treated according to a number of different treatment protocols or modalities. For instance, the horse may be treated using the orthosis 10 described above, underwater treadmill work, stem cells, etc. The wearable device 1600 (which may be incorporated into the orthosis 10 or the sleeve 1500 and worn underneath the orthosis 10) may generate data corresponding to the horse's progress as the horse is rehabilitated. The wearable device 1600 may communicate the sensor data to the performance analytics system 1624. The performance identifier 1636 may receive the data, and may plot the data over time. The performance identifier 1636 may determine, based on trends in the performance over time, whether particular treatment protocols are more effective on the horse as compared to other treatment protocols. For instance, the data may show that the orthosis 10 is resulting in improved performance. The data may also show that underwater treadmill treatment is not effective for the horse's rehabilitation (e.g., no improvement from the underwater training. In some embodiments, the performance identifier 1636 may determine, based on such trends, that the treatment or rehabilitation plan is to be modified based on the data. For instance, the performance identifier may determine that the underwater treadmill is to be foregone from the treatment plan because it is not noticeably improving the horse's condition. The notification generator 1634 may generate a notification (which may be communicated via the communication interface 1626) for a trainer portal or veterinarian portal which indicates the modification to the treatment plan. Continuing the previous example, the trainer or veterinarian may therefore forego underwater treadmill treatment and focus on orthosis 10 treatment. Such embodiments may result in decreased rehabilitation time.

In some embodiments, the performance identifier 1636 may include or access a generic recommended program. For instance, the generic recommended program may include a walking and trotting exercise given in hand or with the use of a horse walker. The surfaces on which the horse is exercised may be selected based on the type of exercise (e.g., softer surfaces when trotting and cantering). In some embodiments, the performance identifier 1636 may modify the generic recommended program (e.g., shortened or lengthened, types of training or exercise, etc.) based on the horse's discipline (e.g., upper level event horses and dressage horses may need more time, while showjumpers could sometimes be rehabilitated more rapidly), the severity of the disease, the structure affected (i.e. DDFT disease might follow the same program, while desmitis of the accessory ligament of the deep digital flexor tendon and the suspensory ligament may complete the same program within less time).

In some embodiments, the performance identifier 1636 may generate a rehabilitation program in accordance with three general phases: a sub-acute phase, an acute phase, and a chronic phase.

In the sub-acute phase, the performance identifier 1636 may identify, select, or otherwise generate a program including rehabilitation that focuses on protecting the injured tissues from anything more than essential movement, to allow healing and control of the early inflammatory process. In some embodiments, the performance identifier 1636 may identify rehabilitation that includes ice, non-steroidal anti-inflammatory drugs which can help reduce inflammation and pain, a leg wrap of the affected limb which may speed dissolution of swelling, etc.

In the acute phase, the performance identifier 1636 may generate a program including rehabilitation that focuses on appropriately increasing the load on the tendon and its muscle through a graduated exercise regimen to provide proper stimuli for healing and the greatest likelihood of an optimal functional outcome. Given that the injured tissue is still at a very sensitive stage in the healing process, the performance identifier 1636 may select rehabilitations which avoid or decrease the likelihood of re-injury of damaged tissues (such as light exercise while continuing various rehabilitation from the sub-acute phase, limited stress and workout, etc.).

In the chronic phase, the performance identifier 1636 may generate a program including rehabilitation that focuses on transitioning motion of the horse from protected (limited) motion to full range of motion. The chronic phase may restore maximal performance and minimizing the risk of re-injury. The performance identifier 1636 may select rehabilitations which focus on restoring strength and flexibility (although inflexibility might be slow to resolve). The performance identifier 1636 may track progress of the horse via the sensors described above. The performance identifier 1636 may gradually add sport-specific exercise as strength of the horse is deemed adequate.

The systems and methods described herein may increase rehabilitation success and decrease rehabilitation time. The performance identifier 1636 may track and assess the rehabilitation of the horse. The performance identifier 1636 may detect forces on the fetlock joint, which may be used for reducing inflammation by reducing inflammatory moieties (e.g., Substance P) which are promoted by flexor loading. The performance identifier 1636 may select or modify the maximum allowable rotation angle of the fetlock joint to reduce inflammation, pain, transfer loads, increase joint and soft tissue range of motion, and reduce the risk of re-injury.

Similarly, the data may be used for improving or optimizing training regimens for a horse. As the horse trains (e.g., using a number of different training regimens), the progress of the horse may be tracked via the wearable device 1600 and corresponding sensor(s) 1610. The sensor(s) 1610 may generate data corresponding to each training regimen. The wearable device 1600 may communicate the sensor data to the performance analytics system 1624. The performance identifier 1636 may analyze the sensor data to determine which types of training are most effective for improving the performance of the horse. In some embodiments, the data may show the horse is most effective at training at certain times of the day (e.g., morning versus the afternoon or evening, for instance). The performance identifier 1636 may identify trends in the data to determine optimal time of day for training. The notification generator 1634 may generate a notification for a veterinarian portal or trainer portal for modifying the time of day for training the horse. In some embodiments, the data may be analyzed to determine an effective duration of training. For instance, the data may show that the horse's performance begins to decrease after a certain duration, which may indicate over-training of the horse. The performance identifier 1636 may identify trends in the data to determine optimal duration for training. The notification generator 1634 may generate a notification for a veterinarian portal or trainer portal for modifying the duration of training the horse.

In each of these instances and examples, the horse's training regimen may be modified based on data generated by the wearable device 1600. The training regimen may be optimized to maximize performance of the horse. Certain training methods may be removed from the training regimen, certain training methods may be added to the training regimen, and the time (and duration) of the training regimen may be modified based on the data. Such embodiments may result in improved performance of the horse.

### E. Incorporation of 3^{rd} Party Data in the Performance Analytics System

In some embodiments, various third-party data may be provided to or otherwise accessed by the performance analytics system 1624. The third-party data may be used by the performance analytics system 1624 for grouping horses (e.g., by the baseline generator 1628), providing further metrics for evaluating horses (e.g., by the performance identifier 1636), etc. In some embodiments, the third-party data may include weather data. In some embodiments, the weather data may be provided to the performance analytics system 1624 by various remote sources, such as The Weather Channel, National Weather Service, Weather Underground ®, Dark Sky ®, or other weather provider. The weather data may indicate rainy conditions, cold conditions, etc. for a particular location associated with the location of the horse (as provided by the positioning sensor(s) 1614, manually provided by an owner/veterinarian/trainer/etc.). Such weather data may be used by the performance identifier 1636 for evaluating or tracking a horses performance in particular weather conditions.

In some embodiments, the weather data may be used by the performance identifier to infer track or path conditions. For instance, the weather data may indicate that it is currently raining at the track or path on which the horse is exercising (based on location data from the positioning sensor(s) 1614). The performance identifier 1636 may infer, based on the weather conditions, that the track or path is muddy. In some embodiments, various track sensors may be used for evaluating track conditions. In still other embodiments, a trainer or jockey may input (e.g., on a portal associated therewith) the type of track (e.g., grass, dirt, sand, etc.). The performance identifier 1636 may tag the sensor data with the track conditions. In some embodiments, the baseline generator 1628 may generate a baseline for the horse (or a group of horses including the horse) based on the tagged sensor data. The baseline may correspond to the particular track positions.

The performance identifier 1636 may separate sensor data (e.g., plotted sensor data) over time by track conditions for tracking a horse's performance over time in particular conditions. In some embodiments, following tracking the horse's performance over time in particular track or weather conditions, the performance identifier 1636 may generate recommendations for modifying training of the horse. The performance identifier 1636 may identify particular training or rehabilitation modalities that are more effective in particular conditions based on trends identified in those specific conditions. The performance identifier 1636 may recommend changes to the training or rehabilitation based on the trends.

In some embodiments, various third-party information pertaining to the horse may be provided to the performance analytics system 1624. For instance, such third-party information pertaining to the horse may include diet, medication, supplements, therapeutic or training modalities, etc. The performance identifier 1636 may use such third-party information for evaluating the effectiveness on the horse. For instance, where a horse is eating a particular food (or taking a particular medication or supplement) and becomes agitated that night, the horse's diet (or medication/supplements) may subsequently be changed. As another example, where a horse has recently begun stem cell treatment and subsequently improves treatment, the stem cell treatment may be attributed to the improved performance and may be used in the future for treatment. The performance identifier 1636 may receive sensor data from the wearable device 1600 corresponding to the horse's vitals, performance, etc. The performance identifier 1636 may identify trends (such as improvements, digressions, or other changes) in the data. The performance identifier 1636 may determine recent changes, such as dietary changes, training changes, rehabilitation changes, etc. The performance identifier 1636 may associate the recent changes to the identified trends. Where the trend is a negative trend (e.g., a decrease in performance over time, for instance), the performance identifier 1636 may recommend removing the recent change. Where the trend is a positive trend (e.g., an increase in performance over time, for instance), the performance identifier 1636 may recommend maintaining, continuing, or expanding on the recent change. Such embodiments may improve the performance of the horse by correlating improvements in performance with potential causes.

In some embodiments, various third-party information pertaining to the jockey/trainer may be provided to the performance analytics system 1624. For instance, identification of the jockey/trainer, height/weight of the jockey, etc. may be provided to the performance analytics system 1624. Such third-party information may be used by the performance identifier 1636 for evaluating the effectiveness of particular jockeys, trainers, etc. Additionally, the height and weight of the jockey may be used by the performance identifier 1636 for determining whether, for instance, an increase in force or change in gait is a result of the jockey (e.g., having a greater weight, for instance) or the horse.

### F. Communications System

In each of the above-mentioned embodiments, a wearable device 1600 collects various information from a horse. Such information is collected by the wearable device 1600 and provided to a performance analytics system 1624. The performance analytics system 1624 uses such data for evaluating the horse, evaluating a training regimen for the horse, optimizing performance of the horse, and decreasing rehabilitation time for the horse. Generally speaking, the data is provided to interested parties with respect to the horse for analysis and modification of treatment, training, exercise, and for diagnosing conditions.

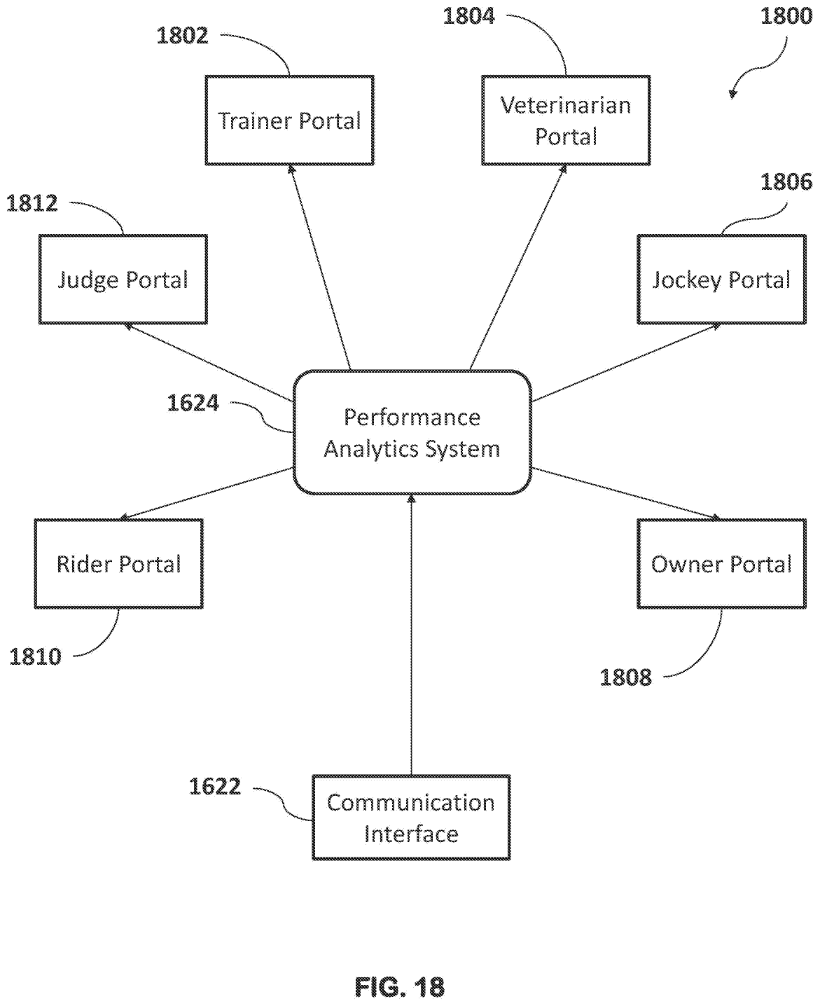

Referring now to FIG. 18, a communications system 1800 for providing horse-related data to interested parties is shown, according to an exemplary embodiment. As shown, the communications system 1800 includes the communication interface 1622 of the wearable device 1600. The communications system 1800 also includes the performance analytics system 1624. The communications system 1800 also includes one or more portals. The portals may be computers, terminals, mobile devices, portable electronic devices, etc., associated with various parties. Each portal may include an associated communication interface. The communication interface for each portal may be communicably coupled to the communication interface 1626 of the performance analytics system. The communication interfaces may be communicably coupled via a computer network. The computer network may be a Local Area Network (LAN), a Wide Area Network (WAN), a Wireless Local Area Network (WLAN), an Internet Area Network (IAN), a cloud-based network, etc. In some implementations, the communication interface 1626 may access the computer network to exchange data with various other communications device via cellular access, a modem, broadband, Wi-Fi, Bluetooth, satellite access, etc. The communication interface 1626 may provide data to the portals upon request, at intervals, and/or in real-time. In some embodiments, a portal may request an update by communicating a signal to the communication interface 1626. The communication interface 1626 may provide the update with the corresponding sensor data to the requesting portal. Hence, the communication interface 1626 may exchange data with the portals.

In some embodiments, the portals may include a trainer portal 1802, a veterinarian portal 1804, a jockey portal 1806, an owner portal 1808, a rider portal 1810, and a judge portal 1812. Each portal may be associated with a trainer, a veterinarian, a jockey, an owner, a rider, and a judge associated with a particular horse. Each person may log into the portal by providing log-in credentials. Each person may register with the horse (e.g., by providing registration information associated with the horse, the wearable devices 1600, etc.). Following the user logging into the portal, the portal may be associated with the horse. The profile manager 1630 may receive the log-in information and an identifier for the portal (e.g., an IP address, for instance). The profile manager 1630 may associate the portal with a particular profile for a horse (including wearable devices 1600 associated with that horse). Each person may therefore receive information from the wearable devices 1600. Such embodiments may provide for increased communication and awareness of the condition of the horse for all interested parties.

In embodiments where a judge portal 1812 is provided in the communication system 1800, the judge may be provided (e.g., via the judge portal 1812) with real-time data from the wearable device 1600. The real-time data may show a horse's performance in a competition. For instance, a judge may be judging how a horse jumps, how straight a horse is during dressage, posture, or other positions. The wearable device 1600 may provide data corresponding to such performance during the competition to the judge. The judge may thus be provided real-time quantitative data for assessing and evaluating the horse in addition to or instead of current qualitative assessments.

The communications system 1800 may provide for cross-communications between interested parties for a horse. In some embodiments, the notification generator 1634 may generate targeted notifications for particular users (e.g., owner-specific notifications, veterinarian and owner-specific notifications, etc.). The notification generator 1634 may dispatch such notifications to each targeted user. Such embodiments may keep, for instance, parties in the loop on decisions or changes for the horse, performance of the horse, location of the horse (e.g., in real-time, when the horse exits a defined area or space, and so forth), etc. In some embodiments, the performance identifier and/or notification generator 1636, 1634 may generate or compile data over a period of time for forming a report for a horse. Such a report may be communicated to each party such that each party may review and interpret the report, and may be informed of the horse's progress/performance.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the orthosis 10, the sleeve 1500, and the wearable device 1600 as shown in the various exemplary embodiments is illustrative only. Additionally, any element disclosed in one embodiment may be incorporated or utilized with any other embodiment disclosed herein.

What is claimed is:

1. A performance analytics system for monitoring a performance of a horse, comprising one or more servers configured to:
receive, via a computing device, sensor data from one or more sensors attached to one or more fetlock wearable devices, each of the one or more fetlock wearable devices configured to attach to a fetlock of a respective limb of a horse;
compare the sensor data to one or more baseline measurement values;
detect a condition responsive to comparing the sensor data to one or more baseline measurement values; and
transmit an alert to one or more remote devices responsive to detecting the condition.

2. The system of claim 1, wherein the one or more baseline measurement values are for a plurality of similarly situated horses.

3. The system of claim 1, wherein the one or more baseline measurement values are for the horse at a previous point in time.

4. The system of claim 1, wherein the fetlock wearable device is a brace including one or more motion restriction elements configured to restrict motion about the fetlock joint.

5. The system of claim 1, wherein the fetlock wearable device is a sleeve including conductive thread.

6. The system of claim 1, wherein the fetlock wearable device includes a sleeve with one or more sensors.

7. The system of claim 1, wherein the condition is at least one of colic or hyper-extension of the fetlock joint.

8. A fetlock wearable device configured to be worn on a limb of a horse, the fetlock wearable device comprising:
one or more sensors attached to the fetlock wearable device;
a communications system communicably coupled to the one or more sensors of the fetlock wearable device and an analytics system, the communications system configured to transmit sensor data from the one or more sensors to the analytics system, wherein the analytics system is configured to:
compare the sensor data to one or more baseline measurement values;
detect a condition responsive to comparing the sensor data to one or more baseline measurement values; and
transmit an alert to one or more remote devices responsive to detecting the condition.

9. The fetlock wearable device of claim 8, wherein the one or more baseline measurement values are for a plurality of similarly situated horses.

10. The fetlock wearable device of claim 8, wherein the one or more baseline measurement values are for the horse at a previous point in time.

11. The fetlock wearable device of claim 8, further comprising:
one or more motion restriction elements configured to restrict motion about the fetlock joint.

12. The fetlock wearable device of claim 8, further comprising:
a sleeve worn around the limb of the horse, the sleeve comprising a conductive thread.

13. The fetlock wearable device of claim 8, further comprising:
a sleeve worn around the limb of the horse, the sleeve comprising the one or more sensors.

14. The fetlock wearable device of claim 8, wherein the condition is at least one of colic or hyper-extension of the fetlock joint.

15. A method for monitoring a performance of a horse, comprising:
receiving, by one or more servers, via a computing device, sensor data from one or more sensors attached to one or more fetlock wearable devices, each of the one or more fetlock wearable devices configured to attach to a fetlock of a respective limb of a horse;
comparing, by the one or more servers, the sensor data to one or more baseline measurement values;
detecting, by the one or more servers, a condition responsive to comparing the sensor data to one or more baseline measurement values; and
transmitting, by the one or more servers, an alert to one or more remote devices responsive to detecting the condition.

16. The method of claim 15, wherein the one or more baseline measurement values are for a plurality of similarly situated horses.

17. The method of claim 15, wherein the one or more baseline measurement values are for the horse at a previous point in time.

18. The method of claim 15, wherein the fetlock wearable device comprises one or more motion restriction elements configured to restrict motion about the fetlock joint.

19. The method of claim 15, wherein the fetlock wearable device comprises a sleeve worn around the limb of the horse, the sleeve comprising at least one of a conductive thread or the one or more sensors.

20. The method of claim 15, wherein the condition is at least one of colic or hyper-extension of the fetlock joint.
